(19) United States
(12) Patent Application Publication
(10) Pub. No.: US 2011/0151564 A1
(43) Pub. Date: Jun. 23, 2011

(54) METHOD FOR PROLIFERATION OF CELLS ON POLYELECTROLYTE MULTILAYER FILMS AND USE THEREOF, PARTICULARLY FOR THE PREPARATION OF CELLULAR BIOMATERIALS

(75) Inventors: Patrick Menu, Vandoeuvre Les Nancy (FR); Cedric Boura, Vandoeuvre Les Nancy (FR); Halima-Assia Kerdjoudj, Vandoeuvre Les Nancy (FR); Vanessa Moby, Dombasle (FR); Nicolas Berthelemy, Nancy (FR); Jean-Claude Voegel, Valff (FR); Pierre Schaaf, Molsheim (FR); Jean-Francois Stoltz, Vandoeuvre Les Nancy (FR)

(73) Assignees: UNIVERSITE HENRI POINCARE NANCY 1, Nancy (FR); CHU DE NANCY-BRABOIS, Vandoeuvre Les Nancy (FR)

(21) Appl. No.: 12/664,907

(22) PCT Filed: Jun. 16, 2008
(86) PCT No.: PCT/FR2008/000832

§ 371 Date: Sep. 3, 2010

(30) Foreign Application Priority Data
Jun. 18, 2007 (FR) 0704313

(51) Int. Cl. C12N 5/07

(52) U.S. Cl. 435/396; 435/395; 435/325

(57) ABSTRACT

The invention relates to the use of a unit including a substrate and polyelectrolyte multilayer films deposited thereon in order to: carry out a method involving the proliferation of initial stem or differentiated cells that are brought into contact with the unit; and cover the unit with confluent viable adherent cells resulting from the proliferation of the initial cells, the cover being obtained at the end of a period of no more than one month, such as 14 days, 11 days or, in particular, 7 days, after the initial cells are brought into contact with the unit.

This invention relates to a method for proliferation of cells on polyelectrolyte multilayer films and use thereof, notably for the preparation of cellular biomaterials.

Polyelectrolytes are polymers whose monomers carry an electrolyte group. These polymers are therefore charged. The layer-by-layer deposition of polyelectrolytes is a simple method for devising surfaces that have special properties [a) G. Decher, J. B. Schlenoff, Multilayer thin films: Sequential Assembly of Nanocomposite Materials, Wiley-VCH, Weinheim, 2003. b) G. Decher, Science 277, 1232, 1997]. By successive immersion or deposition of a substrate alternately in a solution of polyanions and of polycations, an assembly is prepared: substrate and multilayer film of polyelectrolytes, in which the anionic and cationic layers alternate. The driving force of the growth of these multilayer films is the excess charges that appear after each new deposition of a polyelectrolyte and thus permit renewed interaction with the polyelectrolyte of opposite sign. This method of treatment is simple to use, is applicable regardless of the geometry of the substrate and generally only employs aqueous solutions. The physicochemical, viscoelastic, structural, surface roughness and wettability properties of the assembly of substrate and polyelectrolyte multilayer film can be adjusted depending on the required use [A. Izquierdo, S. Ono, J. C. Voegel, P. Schaaf, G. Decher, Langmuir, 21, 7558, 2005].

The use of polyelectrolyte multilayer films makes the functionalization of surfaces possible. Improvement of the interaction between cells and surfaces is important in the fields of medicine, biomaterials and biotechnology.

There are electrostatic interactions between negatively charged substrates (for example glass or expanded polytetrafluoroethylene ePTFE) and cells with an overall negative charge, which is unfavourable for adhesion of cells on these substrates. Formation of a polyelectrolyte multilayer film on a biomaterial can promote cellular adhesion and proliferation.

Polyelectrolyte multilayer films have been used for the proliferation of differentiated cells, for example endothelial cells on a glass slide as substrate [C. Boura, P. Menu, E. Payan, C. Picart, J. C. Voegel, S. Muller, J. F. Stoltz, Biomaterials 24, 3521, 2003] and nerve cells on a substrate of TCPS (polystyrene “treated for cell culture”) [S. Forry, D. Reyes, M. Gaitan, L. Locascio, Langmuir 22, 5770, 2006].

Other techniques are used in the prior art for promoting the adhesion of cells on substrates, in particular covering of the substrates with constituents of the extracellular matrix such as: collagen [H. Itoh, Y. Aso, M. Furuse, Y. Noishiki, T. Miyata, Artif. Organs, 25, 213, 2001], fibronectin [A. Rademacher, M. Paulitschke, R. Meyer, R. Hetzer, Int. J. Artif. Organs, 24, 235, 2001], laminin [A. Sank, K. Rostami, F. Weaver, D. Ertl, A. Yellin, M. Nimni, T. L. Tuan. Am. J. Surg. 164, 199, 1992], gelatin [J. S. Budd, P. R. Bell, R. F. James. Br. J. Surg. 76, 1259, 1989], polylysine [a) J. S. Budd, P. R. Bell, R. F. James, Br. J. Surg. 76, 1259, 1989, b), G. Stansby, N. Shukla, B. Fuller, G. Hamilton. Br. J. Surg. 78, 1189, 1991]. Fibronectin is still the most effective protein for enhancing cellular attachment and retention. Works published following clinical studies have shown considerable hydrolysis of fibronectin, which is rather incompatible with use of this protein in vivo [A. Tiwari, H. J. Salacinski, G. Punshon, G. Hamilton, A. M. Seifalian, FASEB J. 16, 791, 2002]. Improvement of the adhesion of cells on substrates for the preparation of grafts for use in vivo is therefore necessary.

Moreover, the use of these various heterologous constituents (of human origin or often of animal origin) and the need for long proliferation times are sometimes incompatible with therapeutic requirements (for example artificial vessels or skin graft).

Furthermore, the techniques of cellular proliferation for the preparation of grafts are carried out in two stages with the techniques known by a person skilled in the art: a first stage of maturation, proliferation, and differentiation of stem cells and/or the expansion of differentiated cells on a first substrate, then detachment of the cells and seeding on another substrate which will be grafted. The need to use two substrates, and therefore to have to detach and then reseed the cells, is time-consuming and increases the risks of contamination.

One aspect of the invention is to provide a method for proliferation of differentiated or undifferentiated cells, which is quick enough for the preparation of grafts.

One aspect of the invention is to provide a method for proliferation of differentiated or undifferentiated cells, in which the proliferation, the maturation and optionally the differentiation of the cells take place on the same substrate as the one that is to be grafted.

Another aspect of the invention is to provide materials covered with viable cells, such as artificial skin or substitutes for vessels or arteries.

In one of these most general aspects, the invention relates to the use of an assembly comprising a substrate and polyelectrolyte multilayer films deposited on said substrate,
for the application of a method for proliferation of initial, stem or differentiated cells, brought in contact with the aforesaid assembly, and,
covering the aforesaid assembly with adherent, viable and confluent cells resulting from the proliferation of the aforesaid initial cells,
the aforesaid covering being obtained after a period not exceeding one month, notably 14 days, notably 11 days, in particular 7 days, after contacting the aforesaid initial cells with the aforesaid assembly.

In the case of stem cells, the method additionally comprises a stage of differentiation, which also takes place on the aforementioned assembly.

One aspect of the invention relates to the use of polyelectrolyte multilayer films deposited on a substrate, said multilayer films:
being optionally coated, partially or completely, with a collection of biological or biologically active molecules,
and/or optionally comprising biological or biologically active molecules, incorporated between at least two adjacent layers of the aforesaid polyelectrolyte multilayer films, incorporation being such that neither the properties of the polyelectrolyte multilayer films, nor the possible biological properties of said molecules are altered,
* for the application of a method for proliferation of initial, stem or differentiated cells, brought in contact
   with the aforesaid polyelectrolyte multilayer films
   or with the aforesaid collection of biological or biologically active molecules coating the aforesaid polyelectrolyte multilayer films,
* and covering
   the aforesaid polyelectrolyte multilayer films
   or the aforesaid collection of biological or biologically active molecules, with adherent, viable and confluent cells resulting from the proliferation of the aforesaid initial cells,
* the aforesaid covering being obtained after a period not exceeding one month, notably 14 days, notably 11 days, in particular 7 days, after contacting the aforesaid initial cells,
   with the aforesaid polyelectrolyte multilayer films
   or with the aforesaid collection of biological or biologically active molecules.

The invention is based on the finding that the production of a layer of viable, confluent and adherent cells is quicker than with the techniques known by a person skilled in the art.

In the particular case of application of the invention for the preparation of tissues that will be used as grafts (for example substitutes for vessels or arteries), the invention is based on demonstration of the saving in time and money provided by the use of polyelectrolyte multilayer films for the maturation, the proliferation, and the differentiation of stem cells and/or for the proliferation of differentiated cells. In fact, the maturation, proliferation and differentiation of stem cells and/or the proliferation of differentiated cells can be carried out directly on the substrate that will be used for the graft, in contrast to the techniques known by a person skilled in the art, which are carried out in two stages:
maturation, proliferation and differentiation of stem cells and/or proliferation of differentiated cells on a first substrate, then
detachment of the cells and seeding on another substrate, which will be grafted.

“Substrate” means any material on which the layer-by-layer deposition of polyelectrolytes can be carried out.

“Polyelectrolytes” means polymers whose monomers carry an electrolyte group. “Polyelectrolyte multilayer films” means the stack of layers obtained by the layer-by-layer deposition of polyelectrolytes [G. Decher, J. B. Schlenoff, Multilayer thin films: Sequential Assembly of Nanocomposite Materials, Wiley-VCH, Weinheim, 2003].

“Top layer of polyelectrolytes” means the last layer of polyelectrolytes deposited by the technique of layer-by-layer deposition.

“Inner layers of polyelectrolytes” means the layers of polyelectrolytes located between the substrate and the top layer of polyelectrolytes.

“Polycation” means a polymer with an overall positive charge, “with an overall positive charge” meaning that the total charge is positive, but this does not exclude the presence of negatively charged monomers in the polymer.

“Polyanion” means a polymer with an overall negative charge, “with an overall negative charge” meaning that the total charge is negative, but this does not exclude the presence of positively charged monomers in the polymer.

“Biological molecules” means molecules that participate in the metabolic process and in the maintenance of a living organism, for example proteins, DNA, RNA, cytokines, growth factors, for example those necessary for the recruitment and the differentiation of the desired cell type (notably VEGF in the case of the vascular cells).

“Biologically active molecules” means molecules that have curative or preventive properties, for example which accelerate or reduce cell differentiation and/or proliferation, or for example medicinal products (notably VEGF in the case of ischaemia, or taxol in the case of cancers).

The expression “multilayer films coated with an assembly of molecules” denotes that an assembly of molecules is deposited on the surface of the multilayer films. The molecules can be adsorbed on the surface. Interactions occur between the molecules and the top layer of polyelectrolytes, but the molecules can also be buried between the inner polyelectrolyte layers of the multilayer film. For example, in the case when the molecule is a protein with an overall positive charge coating a polyelectrolyte multilayer film whose top layer of polyelectrolytes is a polyanion, the principal electrostatic interactions will be those between the positive charges of the protein and the negative charges of the top layer of polyelectrolytes. However, a protein with an overall positive charge can contain negatively charged amino acids, which do not interact with the top layer of the polyelectrolyte, but instead with the positively charged polyelectrolytes of the inner layer of polyelectrolytes.

The expression “completely coated” means that the molecules coat the entire surface of the polyelectrolyte multilayer film. The expression “partially coated” means that the molecules are only present at certain places on the polyelectrolyte multilayer film. This partial coating can be obtained by spraying techniques, such as those used in the publications [Porcel et al., Langmuir 22, 4376-83, 2006 and Porcel et al., Langmuir 21, 800-02, 2005]. Images obtained with the laser fluorescence microscope or atomic force microscope can make it possible to determine whether the coating is partial or complete.

The expression “comprising biological or biologically active molecules” means that molecules are present in the polyelectrolyte multilayer film. These molecules are incorporated between the layers of polyelectrolytes of the polyelectrolyte multilayer film. The techniques for incorporating molecules between polyelectrolytes are explained in the publications of N. Jessel, M. Oulad-Abdelghani, F. Meyer, P. Lavalle, Y. Haîkel, P. Schaaf, J. C. Voegel, PNAS 103, 8618, 2006 (example of incorporation of a biologically active molecule, β-cyclodextrin) and of A. Dierich, E. Le Guen, N. Messaddeq, J. F. Stoltz, P. Netter, P. Schaaf, J. C. Voegel, N. Benkirane-Jessel, Adv. Mater. 16, 693, 2007 (example of incorporation of growth factors TGFβ₁).

“Adjacent layers” means two layers of polyelectrolytes that were deposited one after another during formation of the polyelectrolyte multilayer film.

“Properties of the polyelectrolyte multilayer film” means the physicochemical properties, notably the viscoelasticity, surface roughness and wettability of the polyelectrolyte multilayer film.

“Biological properties of said molecules” means the curative or preventive properties of the biologically active molecules.

“Proliferation of cells” means the division and maturation of cells.

“Initial cells” means the cells that are brought in contact initially with the polyelectrolyte multilayer film.

“Covering of the multilayer films with cells” means the production of a layer, preferably a monolayer, of cells, deposited on the polyelectrolyte multilayer film. The cells can be adsorbed on the top layer of polyelectrolytes of the multilayer film, but there may also be interactions with inner layers of polyelectrolytes of the polyelectrolyte multilayer film. These interactions can for example be ionic bonds, hydrogen bonds, van der Waals bonds etc.

“Adherent cells” means cells that adhere to the polyelectrolyte multilayer film or to any biological or biologically active molecules with which it is coated. This adhesion can for example be visualized by images of histological sections or from observation with the scanning electron microscope and can be confirmed via the expression of specific markers of the cells (for example, integrins and the arrangement of the cytoskeleton).

“Viable cells” means cells that are capable of surviving. Cell viability can for example be determined by the ABRA test (Alamar Blue® redox assay).

“Confluent cells” means cells whose cell membranes are in contact. This occurs when the initial cells put in culture have proliferated so as to occupy all the available space in a monolayer. Confluence can be detected from images obtained in phase-contrast or laser-scanning microscopy.

“Cells resulting from proliferation of the initial cells” means the cells resulting from the division, maturation, and optionally differentiation (when the initial cells are stem cells) of the initial cells.

Another aspect of the invention relates to the use of polyelectrolyte multilayer films deposited on a substrate, said multilayer films:
being optionally coated, partially or completely, with a collection of biological or biologically active molecules,
and/or optionally comprising biological or biologically active molecules, incorporated between at least two adjacent layers of the aforesaid polyelectrolyte multilayer films, incorporation being such that any chemical bonds between the aforesaid molecules and the layers of polyelectrolytes are not of a covalent nature, for:
* the application of a method for proliferation of initial, stem or differentiated cells, brought in contact
   with the aforesaid polyelectrolyte multilayer films
   or with the aforesaid collection of biological or biologically active molecules coating the aforesaid polyelectrolyte multilayer films,
* and covering
   of the aforesaid polyelectrolyte multilayer films
   or of the aforesaid collection of biological or biologically active molecules, with adherent, viable and confluent cells resulting from the proliferation of the aforesaid initial cells,
* the aforesaid covering being obtained after a period not exceeding one month, notably 14 days, notably 11 days, in particular 7 days, after contacting the aforesaid initial cells,
   with the aforesaid polyelectrolyte multilayer films
   or with the aforesaid collection of biological or biologically active molecules.

The expression “chemical bond not being of a covalent nature” means that the bonds between the molecules and the layers of polyelectrolytes are, for example, ionic bonds, hydrogen bonds, or van der Waals bonds, which do not alter the properties of the molecules and of the polyelectrolyte multilayer film.

According to one aspect of the invention, the top layer of polyelectrolytes of the multilayer films is a polycation and the multilayer films are not coated with a collection of biological or biologically active molecules, and do not contain biological or biologically active molecules incorporated between at least two adjacent layers of the aforesaid polyelectrolyte multilayer films.

When the top layer of polyelectrolytes is positively charged, the cells, whose membrane is negatively charged, generally adhere to the polyelectrolyte multilayer film.

According to another aspect of the invention, the top layer of polyelectrolytes of the multilayer films is a polyanion and the multilayer films are not coated with a collection of biological or biologically active molecules, and do not contain biological or biologically active molecules incorporated between at least two adjacent layers of the aforesaid polyelectrolyte multilayer films.

When the top layer of polyelectrolytes is negatively charged, the cells, whose membrane is negatively charged, generally do not adhere to the polyelectrolyte multilayer film (repulsive electrostatic interactions).

These last two cases correspond to the use of a polyelectrolyte multilayer film for cellular proliferation without intervention of biological or biologically active molecules.

According to one aspect of the invention, the top layer of polyelectrolytes of the multilayer films is a polycation and the multilayer films are coated with a collection of biological or biologically active molecules and optionally contain biological or biologically active molecules incorporated between at least two adjacent layers of the aforesaid polyelectrolyte multilayer films.

According to another aspect of the invention, the top layer of polyelectrolytes of the multilayer films is a polyanion and the multilayer films are coated with a collection of biological or biologically active molecules and optionally contain biological or biologically active molecules incorporated between at least two adjacent layers of the aforesaid polyelectrolyte multilayer films.

These last two aspects relate to different cases:
the multilayer films are coated with a collection of biological or biologically active molecules, and in this case the cells adhere to this collection of molecules, or,
the multilayer films contain biological or biologically active molecules incorporated between at least two adjacent layers, and in this case the cells adhere to the polyelectrolyte multilayer film, or,
the multilayer films are coated with a collection of biological or biologically active molecules and they contain biological or biologically active molecules incorporated between at least two adjacent layers and in this case the cells adhere to said collection of molecules.

When the top layer of polyelectrolytes is negatively charged, and therefore when the cells do not adhere to the multilayer film, coating of the polyelectrolyte multilayer film with biological molecules is particularly advantageous as it can make it possible to reverse the polarity of the substrate and therefore promote adhesion of the cells.

For example, the top layer of a (PAH-PSS)₃ multilayer film is negatively charged and the cells do not generally adhere. If the multilayer film is covered with proteins with an overall positive charge, the polarity of the surface is reversed and adhesion of the cells is promoted.

In the present invention and according to an advantageous embodiment, the initial cells are differentiated cells, notably selected from keratinocytes, chondrocytes, nerve cells, dendritic cells, endothelial cells, fibroblasts, epiblasts, myoblasts, cardiomyoblasts, myocytes, epithelial cells, osteocytes, osteoblasts, hepatocytes and cells of the islets of Langerhans.

According to another embodiment of the invention, the initial cells are stem cells, notably selected from totipotent, pluripotent and multipotent cells.

“Totipotent cells” means cells that can be differentiated into any cell type of the organism. They permit the development of a complete individual.

“Pluripotent cells” means cells that can be differentiated into cells derived from any of the three germ layers. They cannot produce a complete organism.

“Multipotent cells” means cells that can be differentiated into several types of differentiated cells but only for particular types of cells. For example, haematopoietic multipotent cells can differentiate into red blood cells, platelets, lymphocytes or macrophages but they cannot differentiate into muscle cells.

As examples of stem cells, we may mention embryonic and haematopoietic stem cells, mesenchymal cells, precursors such as EPCs (endothelial progenitor cells).

According to another advantageous embodiment of the invention, the polyelectrolyte multilayer films are constituted of alternating layers of polycations and polyanions,
the so-called polycations are notably selected from polyallylamine (PAH), polyethyleneimine (PEI), polyvinylamine, polyaminoamide (PAMAM), polyacrylamide (PAAm), polydiallyldimethylammonium chloride (PDAC), positively charged polypeptides such as polylysine and positively charged polysaccharides such as chitosan,
and the so-called polyanions are notably selected from polyacrylic acid (PAA), polymethacrylic acid (PMA), polystyrene sulphonic acid (polystyrene sulphonate, PSS or sodium polystyrene sulphonate, SPS), negatively charged polypeptides such as polyglutamic acid and polyaspartic acid and negatively charged polysaccharides such as hyaluronan and alginate.

According to another advantageous embodiment of the invention, the number of layers of the polyelectrolyte multilayer films is from 3 to 100, in particular 3 to 50, notably 5 to 10 and in particular 7.

Below 7 layers, the film is still permeable to small molecules, for example to Hoechst 33258 (molecular weight 623 Da).

According to another advantageous embodiment of the invention, the polyelectrolyte multilayer films are selected from (PAH-PSS)₃, (PAH-PSS)₃-PAH and PEI-(PSS-PAH)₃. [a) H. Kerdjoudj et al. Bio-Medical Materials and Engineering, 16(4), 123, 2006 b) C. Boura et al. Biomaterials 26, 4568, 2005].

According to another advantageous embodiment of the invention, the substrate is a synthetic substrate advantageously selected from glass, TCPS (“treated cell culture” polystyrene), polysiloxane, perfluoroalkyl polyethers, biocompatible polymers especially Dacron®, polyurethane, polydimethylsiloxane, polyvinyl chloride, Silastic®, polytetrafluoroethylene (ePTFE), and any material used for prostheses and/or implanted systems.

According to another advantageous embodiment of the invention, the substrate is a natural substrate advantageously selected from blood vessels, veins, arteries, notably decellularized, notably de-endothelialized umbilical arteries, said vessels, veins and arteries being obtained from organs from donors or from animals.

According to another advantageous embodiment of the invention, the substrate is a natural substrate advantageously selected from the placental dermis, the bladder or any other substrate (organ) of human or animal origin.

According to an advantageous embodiment of the present invention, the polyelectrolyte multilayer films deposited on a substrate are sufficiently rigid to permit the adhesion of cells and sufficiently flexible to deform under the action of arterial pulsations and withstand physiological pressures from 10 to 300 mmHg, notably 50 to 250 mmHg and advantageously 80 to 230 mmHg.

This pressure range corresponds to that observed for physiological pressures. In humans, hypertension is said to be severe if the systolic pressure is above 180 mmHg Hypotension refers to systolic pressure below 50 mmHg.

“Physiological pressures” means the pressures of the blood in the arteries, veins and vessels in a healthy subject.

According to an advantageous embodiment of the present invention, the covering of the polyelectrolyte multilayer films deposited on the substrate with the adherent cells is such that it withstands the shearing action of the blood flow, notably in vivo.

“Shearing action of the blood flow” means the frictional tangential force induced by the blood flow that is exerted on the polyelectrolyte multilayer film when the assembly: substrate, polyelectrolyte multilayer film, and cells covering it, is in physiological conditions.

According to another advantageous embodiment, the invention makes it possible to prepare vascular endoprostheses, balloons for angioplasty, artificial arteries or vessels for grafts, vascular shunts, heart valves, artificial components for the heart, pacemakers, ventricular assist devices, catheters, contact lenses, intraocular lenses, matrices for tissue engineering, biomedical membranes, dialysis membranes, membranes for cell encapsulation, prostheses for cosmetic surgery, orthopaedic prostheses, dental prostheses, dressings, sutures, diagnostic biosensors.

The invention also relates to a method of covering initial cells, stem cells or differentiated cells, comprising:
bringing initial cells in contact with polyelectrolyte multilayer films deposited on a substrate, said multilayer films being optionally coated with a collection of biological or biologically active molecules and/or optionally containing biological or biologically active molecules, incorporated between at least two adjacent layers of the aforesaid polyelectrolyte multilayer films, the incorporation being such that neither the properties of the polyelectrolyte multilayer film, nor the possible biological properties of said molecules are altered, and preferably such that any chemical bonds between the aforesaid molecules and the layers of polyelectrolytes are not of a covalent nature, in media permitting the proliferation of said initial cells,
the proliferation of the aforesaid initial cells,
obtaining, after a period not exceeding one month, notably 14 days, notably 11 days, in particular 7 days, after the aforesaid contacting, covering of the aforesaid multilayer films or of the aforesaid collection of molecules coating the multilayer films, with adherent, viable and confluent cells resulting from the proliferation of the aforesaid initial cells,
optional recovery of said adherent, viable and confluent cells.

At the end of the process, the cells may or may not be detached from the polyelectrolyte multilayer film. For example, for the preparation of artificial skin, the cells will be detached from the multilayer film. Conversely, for the preparation of vascular or arterial substitutes, the endothelial cells are not detached, provided that the substrate is biocompatible, since the assembly: biocompatible substrate/polyelectrolyte multilayer film/endothelial cells, is grafted.

“Biocompatible substrate” means a substrate that is well tolerated by a living organism, which does not cause rejection, toxic reactions, lesions or a harmful effect on the biological functions of the organism.

According to an advantageous embodiment of the present invention, the method is a method of covering initial stem cells comprising:
bringing initial stem cells in contact with polyelectrolyte multilayer films deposited on a substrate in media permitting the proliferation of said initial cells,
the proliferation of the aforesaid stem cells
the maturation and differentiation of the aforesaid stem cells into differentiated cells,
proliferation of the aforesaid differentiated cells derived from the aforesaid initial cells,
obtaining, after a period not exceeding one month, notably 14 days, after the aforesaid contacting, covering of the aforesaid multilayer films with adherent, viable and confluent cells resulting from the proliferation of the aforesaid initial cells,
optional recovery of said adherent, viable and confluent cells.

In this case the initial cells are stem cells. It was found, unexpectedly, that the stem cells can proliferate and differentiate up to confluence in a shorter time than in the methods of the prior art. The time taken in the invention is 14 days, notably 11 days, in particular 7 days. For example, on a glass slide substrate and with the (PAH-PSS)₃-PAH polyelectrolyte multilayer film, confluence is reached in 14 days whereas it takes 60 days when using fibronectin (which is the protein giving the quickest proliferation and differentiation times among the techniques known by a person skilled in the art).

According to an advantageous embodiment of the present invention, the method is a method of covering differentiated initial cells comprising:
bringing differentiated initial cells in contact with polyelectrolyte multilayer films deposited on a substrate in media permitting the proliferation of said initial cells,
the proliferation of the aforesaid differentiated cells,
obtaining, after a period not exceeding one month, notably 7 days, in particular 3 days after the aforesaid contacting, covering of the aforesaid multilayer films with adherent, viable and confluent cells resulting from the proliferation of the aforesaid initial cells,
optional recovery of said adherent, viable and confluent cells.

In this case the initial cells are differentiated cells. It was found, unexpectedly, that the initial cells can proliferate up to confluence in a shorter time than in the methods of the prior art. The time taken in the invention is 7 days, notably 5 days, in particular 3 days. For example, on the ePTFE substrate and with the PEI-(PSS-PAH)₃ polyelectrolyte multilayer film, confluence is reached in 7 days or less, whereas without deposition of a polyelectrolyte multilayer film, no cells adhere.

According to an advantageous embodiment, in the method of the invention the multilayer films are coated with a collection of biological or biologically active molecules, and/or contain biological or biologically active molecules, incorporated between at least two adjacent layers of the aforesaid polyelectrolyte multilayer films, the incorporation being such that neither the properties of the polyelectrolyte multilayer film, nor the possible biological properties of said molecules are altered.

According to an advantageous embodiment of the present invention, the method comprises:
bringing initial cells in contact with polyelectrolyte multilayer films deposited on a substrate, said multilayer films being optionally coated with a collection of biological or biologically active molecules, and/or optionally containing biological or biologically active molecules, incorporated between at least two adjacent layers of the aforesaid polyelectrolyte multilayer films, the incorporation being such that neither the properties of the polyelectrolyte multilayer film, nor the possible biological properties of said molecules are altered, and preferably such that any chemical bonds between the aforesaid molecules and the layers of polyelectrolytes are not of a covalent nature, in media permitting the proliferation of said initial cells,
the proliferation of the aforesaid initial cells,
obtaining, after a period not exceeding one month, notably 14 days, notably 11 days, in particular 7 days, after the aforesaid contacting, covering of the aforesaid multilayer films or of the aforesaid collection of biological or biologically active molecules coating the multilayer film, with adherent, viable and confluent cells resulting from the proliferation of the aforesaid initial cells,
recovery of said adherent, viable and confluent cells.

In this case, the adherent, viable and confluent cells are detached from the polyelectrolyte multilayer film.

According to an advantageous embodiment of the present invention, the method comprises:
bringing initial cells in contact with polyelectrolyte multilayer films deposited on a substrate, said multilayer films being optionally coated with a collection of biological or biologically active molecules, and/or optionally containing biological or biologically active molecules, incorporated between at least two adjacent layers of the aforesaid polyelectrolyte multilayer films, the incorporation being such that neither the properties of the polyelectrolyte multilayer film, nor the possible biological properties of said molecules are altered, and preferably such that any chemical bonds between the aforesaid molecules and the layers of polyelectrolytes are not of a covalent nature, in media permitting the proliferation of said initial cells,
the proliferation of the aforesaid initial cells,
obtaining, after a period not exceeding one month, notably 14 days, notably 11 days, after the aforesaid contacting, covering of the aforesaid multilayer films or of the aforesaid collection of biological or biologically active molecules coating the multilayer films, with adherent, viable and confluent cells resulting from the proliferation of the aforesaid initial cells.

In this case, the adherent, viable and confluent cells are not detached from the polyelectrolyte multilayer film.

According to a preferred embodiment, the method is a method of covering endothelial initial cells which comprises:
bringing endothelial initial cells in contact with polyelectrolyte multilayer films selected from (PAH-PSS)₃, (PAH-PSS)₃-PAH and PEI-(PSS-PAH)₃, deposited on a substrate, notably a natural substrate such as a decellularized, notably de-endothelialized, blood vessel or artery, or a biocompatible synthetic substrate having the shape of a vessel or artery, said multilayer films being optionally coated with a collection of biological or biologically active molecules, and/or optionally containing biological or biologically active molecules, incorporated between at least two adjacent layers of the aforesaid polyelectrolyte multilayer films, the incorporation being such that neither the properties of the polyelectrolyte multilayer film, nor the possible biological properties of said molecules are altered, and preferably such that any chemical bonds between the aforesaid molecules and the layers of polyelectrolytes are not of a covalent nature, in media permitting the proliferation of the aforesaid endothelial initial cells,
the proliferation of the aforesaid endothelial initial cells,
obtaining, after a period not exceeding one month, notably 7 days, notably 5 days, in particular 3 days, after the aforesaid contacting, covering of the aforesaid multilayer films or of the aforesaid collection of biological or biologically active molecules coating the multilayer film, with adherent, viable and confluent endothelial cells resulting from the proliferation of the aforesaid endothelial initial cells.

This case corresponds to a method for proliferation of endothelial cells on a polyelectrolyte multilayer film for the preparation of vascular or arterial substitutes which will be used as grafts. The use of polyelectrolyte multilayer films offers many advantages. Thus, the assembly of artery or vessel substrate/polyelectrolyte multilayer film is sufficiently rigid to permit adhesion of the cells and sufficiently elastic to withstand the deformation caused by the blood flow. Moreover, the monolayer of cells obtained must allow the passage of oxygen and nutrients, which should permit the essential exchanges between the blood and the surrounding tissues.

According to another preferred embodiment, the method is a method of covering initial stem cells comprising:
bringing initial stem cells in contact with polyelectrolyte multilayer films selected from (PAH-PSS)₃, (PAH-PSS)₃-PAH and PEI-(PSS-PAH)₃, deposited on a substrate, said multilayer films being optionally coated with a collection of biological or biologically active molecules, and/or optionally containing biological or biologically active molecules, incorporated between at least two adjacent layers of the aforesaid polyelectrolyte multilayer films, the incorporation being such that neither the properties of the polyelectrolyte multilayer film, nor the possible biological properties of said molecules are altered, and preferably such that any chemical bonds between the aforesaid molecules and the layers of polyelectrolytes are not of a covalent nature, in media permitting the proliferation of the aforesaid initial stem cells,
the proliferation of the aforesaid initial stem cells,
the maturation and differentiation of the aforesaid stem cells into endothelial cells,
the proliferation of the aforesaid endothelial cells derived from the aforesaid initial stem cells,
obtaining, after a period not exceeding 14 days after the aforesaid contacting, covering of the aforesaid multilayer films or of the aforesaid collection of biological or biologically active molecules coating the multilayer film, with adherent, viable and confluent endothelial cells resulting from the proliferation of the aforesaid initial stem cells.

This case corresponds to a method of proliferation of stem cells, then differentiation into endothelial cells, on a polyelectrolyte multilayer film for the preparation of vascular or arterial substitutes which will be used as grafts.

In the method of the invention, the initial stem cells are notably selected from totipotent, pluripotent and multipotent cells.

In the method of the invention, the differentiated initial cells are notably selected from keratinocytes, chondrocytes, nerve cells, dendritic cells, endothelial cells, fibroblasts, epiblasts, myoblasts, cardiomyoblasts, myocytes, epithelial cells, osteocytes, osteoblasts, hepatocytes and cells of the islets of Langerhans.

According to a particular embodiment, in the method of the invention, the polyelectrolyte multilayer films are constituted of layers, preferably alternating, of polycations and of polyanions,
the polycations notably being selected from polyallylamine (PAH), polyethyleneimine (PEI), polyvinylamine, polyaminoamide (PAMAM), polyacrylamide (PAAm), polydiallyldimethylammonium chloride (PDAC), positively charged polypeptides such as polylysine and positively charged polysaccharides such as chitosan,
and the polyanions notably being selected from polyacrylic acid (PAA), polymethacrylic acid (PMA), polystyrene sulphonic acid (PSS or SPS), negatively charged polypeptides such as polyglutamic acid and polyaspartic acid and negatively charged polysaccharides such as hyaluronan and alginate.

Advantageously, the number of layers of said polyelectrolyte multilayer films is from 3 to 100, in particular 3 to 50, notably 5 to 10 and in particular 7.

According to another embodiment of the invention, in the method of the invention the polyelectrolyte multilayer films are selected from (PAH-PSS)₃, (PAH-PSS)₃-PAH and PEI-(PSS-PAH)₃.

According to another embodiment of the invention, in the method of the invention the substrate is selected from synthetic substrates such as glass, TCPS (“treated cell culture” polystyrene), polysiloxane, perfluoroalkyl polyethers, biocompatible polymers especially Dacron®, polyurethane, polydimethylsiloxane, polyvinyl chloride, Silastic®, polytetrafluoroethylene (ePTFE) and any material used for prostheses and/or implanted systems.

According to another embodiment of the invention, in the method of the invention the substrate is selected from natural substrates such as blood vessels, veins, arteries, notably decellularized, notably de-endothelialized umbilical arteries, said vessels, veins and arteries being obtained from organs of donors or of animals.

According to another advantageous embodiment of the invention, the substrate is a natural substrate advantageously selected from the placental dermis, the bladder or any other substrate (organ) of human or animal origin.

The present invention relates to a composition comprising:
a substrate,
polyelectrolyte multilayer films deposited on said substrate, said multilayer films being optionally coated with a collection of biological or biologically active molecules and/or optionally containing biological or biologically active molecules, incorporated between at least two adjacent layers of the aforesaid polyelectrolyte multilayer films, the incorporation being such that neither the properties of the polyelectrolyte multilayer film, nor the possible biological properties of said molecules are altered, and preferably such that any chemical bonds between the aforesaid molecules and the layers of polyelectrolytes are not of a covalent nature, and,
a layer of stem cells covering said polyelectrolyte multilayer films.

According to another embodiment, the composition of the invention comprises a substrate, polyelectrolyte multilayer films deposited on said substrate, said multilayer films are coated with a collection of biological or biologically active molecules and/or contain biological or biologically active molecules, incorporated between at least two adjacent layers of the aforesaid polyelectrolyte multilayer films.

According to another embodiment, the composition of the invention comprises a substrate, polyelectrolyte multilayer films deposited on said substrate, and a layer of stem cells covering said polyelectrolyte multilayer film.

According to a particular embodiment, the initial stem cells are notably selected from totipotent, pluripotent and multipotent cells.

According to another embodiment, the composition of the invention comprises:
a natural substrate,
polyelectrolyte multilayer films deposited on said substrate, said multilayer films being optionally coated with a collection of biological or biologically active molecules and/or optionally containing biological or biologically active molecules, incorporated between at least two adjacent layers of the aforesaid polyelectrolyte multilayer films, the incorporation being such that neither the properties of the polyelectrolyte multilayer film, nor the possible biological properties of said molecules are altered, and preferably such that any chemical bonds between the aforesaid molecules and the layers of polyelectrolytes are not of a covalent nature, and,
a layer of differentiated cells covering said polyelectrolyte multilayer film.

According to an advantageous embodiment, the composition of the invention comprises:
a natural substrate,
polyelectrolyte multilayer films deposited on said substrate, said multilayer films being coated with a collection of biological or biologically active molecules and/or containing biological or biologically active molecules, incorporated between at least two adjacent layers of the aforesaid polyelectrolyte multilayer films, the incorporation being such that neither the properties of the polyelectrolyte multilayer film, nor the possible biological properties of said molecules are altered, and preferably such that any chemical bonds between the aforesaid molecules and the layers of polyelectrolytes are not of a covalent nature, and,
a layer of differentiated cells covering said biological or biologically active molecules.

According to another advantageous embodiment, the composition of the invention comprises:
a natural substrate,
polyelectrolyte multilayer films deposited on said substrate, and,
a layer of differentiated cells covering said polyelectrolyte multilayer film.

According to another embodiment, the composition of the invention comprises:
a substrate,
polyelectrolyte multilayer films deposited on said substrate, said multilayer films being coated with a collection of biological or biologically active molecules, and/or containing biological or biologically active molecules, incorporated between at least two adjacent layers of the aforesaid polyelectrolyte multilayer films, the incorporation being such that neither the properties of the polyelectrolyte multilayer film, nor the possible biological properties of said molecules are altered, and preferably such that any chemical bonds between the aforesaid molecules and the layers of polyelectrolytes are not of a covalent nature, and,
a layer of differentiated cells covering said biological or biologically active molecules.

In the composition of the invention defined above, the substrate is a synthetic or natural substrate, and in particular a synthetic substrate.

In the compositions of the invention, the differentiated initial cells are notably selected from keratinocytes, chondrocytes, nerve cells, dendritic cells, endothelial cells, fibroblasts, epiblasts, myoblasts, cardiomyoblasts, myocytes, epithelial cells, osteocytes, osteoblasts, hepatocytes and cells of the islets of Langerhans.

In the compositions of the invention, the polyelectrolyte multilayer films are constituted of layers, preferably alternating, of polycations and of polyanions,
the polycations notably being selected from polyallylamine (PAH), polyethyleneimine (PEI), polyvinylamine, polyaminoamide (PAMAM), polyacrylamide (PAAm), polydiallyldimethylammonium chloride (PDAC), positively charged polypeptides such as polylysine and positively charged polysaccharides such as chitosan,
and the polyanions notably being selected from polyacrylic acid (PAA), polymethacrylic acid (PMA), polystyrene sulphonic acid (PSS or SPS), negatively charged polypeptides such as polyglutamic acid and polyaspartic acid and negatively charged polysaccharides such as hyaluronan and alginate.

According to an advantageous embodiment, in the compositions of the invention the number of layers of said polyelectrolyte multilayer films is from 3 to 100, in particular 3 to 50, notably 5 to 10 and in particular 7.

According to an advantageous embodiment, in the compositions of the invention the polyelectrolyte multilayer films are selected from (PAH-PSS)₃, (PAH-PSS)₃-PAH and PEI-(PSS-PAH)₃.

In the compositions of the invention, the substrate is selected from natural substrates, such as blood vessels, veins, arteries, notably decellularized, notably de-endothelialized umbilical arteries, said vessels, veins and arteries being obtained from organs of donors or of animals, and the placental dermis. (idem)

In the compositions of the invention, the substrate is advantageously selected from the placental dermis, the bladder or any other substrate (organ) of human or animal origin.

In the compositions of the invention, the substrate is selected from synthetic substrates, notably glass, TCPS (“treated cell culture” polystyrene), polysiloxane, perfluoroalkyl polyethers, biocompatible polymers especially Dacron®, polyurethane, polydimethylsiloxane, polyvinyl chloride, Silastic®, polytetrafluoroethylene (ePTFE) and any material used for prostheses and/or implanted systems.

### FIGURE CAPTIONS

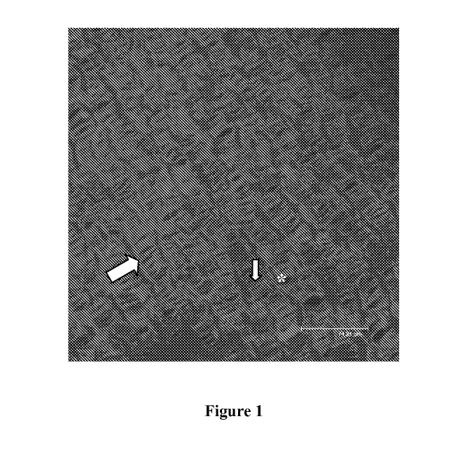

### FIG. 1:

FIG. 1 shows an image obtained with a confocal microscope, objective 40, of an ePTFE substrate on which the [PEI-(PSS-PAH)₂-PSS-PAH*] polyelectrolyte multilayer film was deposited, PAH* being poly(allylamine) hydrochloride coupled to rhodamine.
The microscope is a Leica SP2-AOBS microscope (objective: ×40, ON=0.8, Germany).
The small arrow corresponds to a microfibril or to the distance between two nodes, and the large arrow corresponds to the nodes. The asterisk corresponds to a pore.

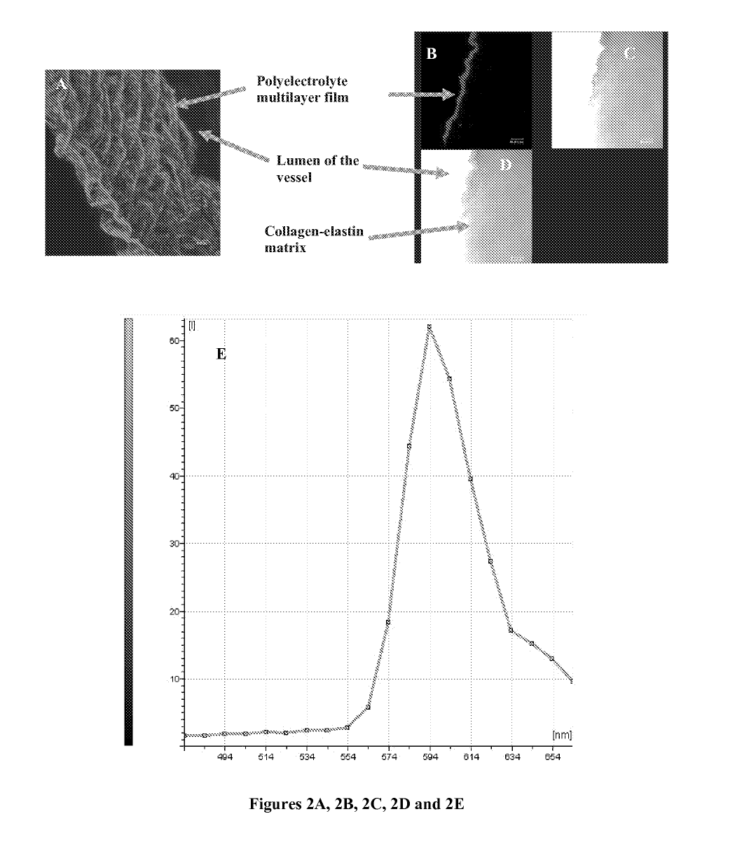

### FIGS. 2A, 2B, 2C, 2D and 2E:

FIG. 2A shows an image obtained with a confocal microscope, objective 40, (n=4) of an artery on which the [(PAH-PSS)₂-PAH*-PSS-PAH*] polyelectrolyte multilayer film was deposited, PAH* being poly(allylamine) hydrochloride (PAH) coupled to rhodamine. This image shows the topology of the internal surface of the artery.

FIG. 2B shows an image obtained with a confocal microscope, objective 40, (n=4) of an artery on which the [(PAH-PSS)₂-PAH*-PSS-PAH*] polyelectrolyte multilayer film was deposited, PAH* being poly(allylamine) hydrochloride (PAH) coupled to rhodamine. This image is a cross-section and shows that covering with the polyelectrolyte multilayer film has occurred on the entire internal surface of the artery.

FIG. 2C shows an image obtained with a confocal microscope, objective 40, (n=4) of an umbilical artery in transmitted light.

FIG. 2D is a superposition of FIGS. 2B and 2C.

FIG. 2E shows the spectrum of rhodamine, confirming the presence of the [(PAH-PSS)₂-PAH*-PSS-PAH*] polyelectrolyte multilayer film. The wavelength in nanometres is shown on the abscissa. The luminosity expressed in grey levels is shown on the ordinate.

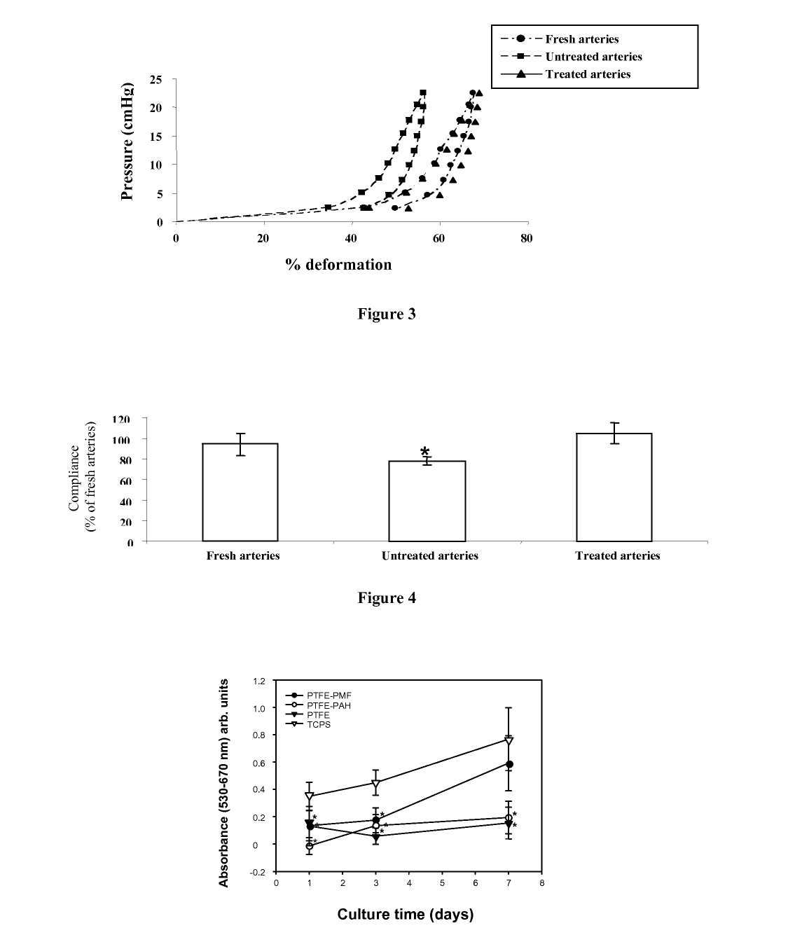

### FIG. 3:

FIG. 3 shows curves of deformation of the arteries as a function of the pressure exerted in said arteries.
The pressure in the arteries in cmHg is shown on the ordinate.
The percentage deformation of the artery is shown on the abscissa.
The curve with dots • represents fresh arteries.
The curve with squares ▪ represents de-endothelialized arteries on which no polyelectrolyte multilayer film was deposited.
The curve with triangles ▴ represents de-endothelialized arteries on which a (PAH-PSS)₃-PAH polyelectrolyte multilayer film was deposited.

### FIG. 4:

FIG. 4 shows the compliance as a percentage relative to fresh arteries, i.e. the data were normalized so that the compliance of fresh arteries is 100%.
The compliance as a percentage relative to fresh arteries is shown on the ordinate.
The type of arteries is shown on the abscissa: fresh arteries, de-endothelialized arteries on which no polyelectrolyte multilayer film was deposited, and de-endothelialized arteries on which a (PAH-PSS)₃-PAH polyelectrolyte multilayer film was deposited.
The symbol * denotes that the compliance of the de-endothelialized arteries on which no polyelectrolyte multilayer film was deposited, differs significantly from that of the fresh arteries with an error probability less than 0.05%.

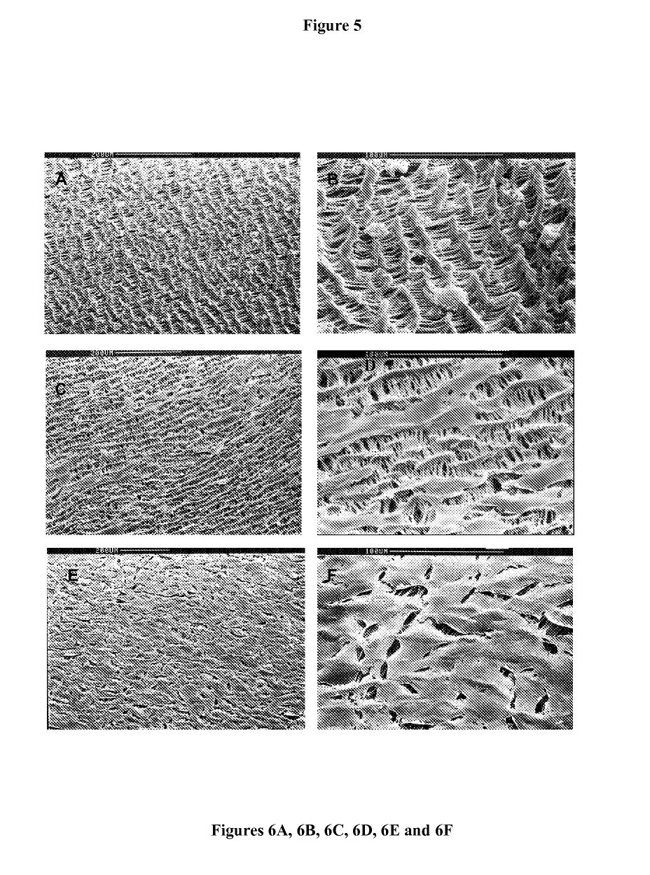

### FIG. 5:

FIG. 5 shows the result of the viability test by the Alamar Blue® assay of endothelial cells HUVECs sown on:
TCPS (curve with empty triangles ∇),
ePTFE (curve with filled triangles ▾),
ePTFE on which the PAH polyelectrolyte was deposited (curve with empty circles ∘), or,
ePTFE on which the PEI-(PSS-PAH)₃ polyelectrolyte multilayer film was deposited (curve with filled circles )
ΔOD=[OD(570 nm)_{exp.}−OD(630 nm)_{exp.}]−[OD(570 nm)_{cont.}−OD(630 nm)_{cont.}] with exp.=experimental, cont.=control without cells and Δ=difference, is shown on the ordinate.
The culture time in days is shown on the abscissa.
The symbol * denotes that the metabolic activity of the endothelial cells on the substrates in question differs significantly from that of the cells on the TCPS substrate with an error probability less than 0.05%.
The incubation time is 3 hours.

### FIG. 6A, 6B, 6C, 6D, 6E and 6F:

FIG. 6A shows the image, observed with an electron microscope (magnification ×169), of the ePTFE substrate on which endothelial cells were cultivated.

FIG. 6B shows the image, observed with an electron microscope (magnification ×508), of the ePTFE substrate on which endothelial cells were cultivated.

FIG. 6C shows the image, observed with an electron microscope (magnification ×149), of the ePTFE substrate on which the PAH polyelectrolyte was deposited and on which endothelial cells were cultivated.

FIG. 6D shows the image, observed with an electron microscope (magnification ×503), of the ePTFE substrate on which the PAH polyelectrolyte was deposited and on which endothelial cells were cultivated.

FIG. 6E shows the image, observed with an electron microscope (magnification ×112), of the ePTFE substrate on which the PEI-(PSS-PAH)₃ polyelectrolyte multilayer film was deposited and on which endothelial cells were cultivated.

FIG. 6F shows the image, observed with an electron microscope (magnification ×513), of the ePTFE substrate on which the PEI-(PSS-PAH)₃ polyelectrolyte multilayer film was deposited and on which endothelial cells were cultivated.

For FIGS. 6A, 6B, 6C, 6D, 6E and 6F, the culture time of the endothelial cells HUVECs is 7 days, and the microscope is a STEREOSCAN S 240 electron microscope, CAMBRIDGE (UK).

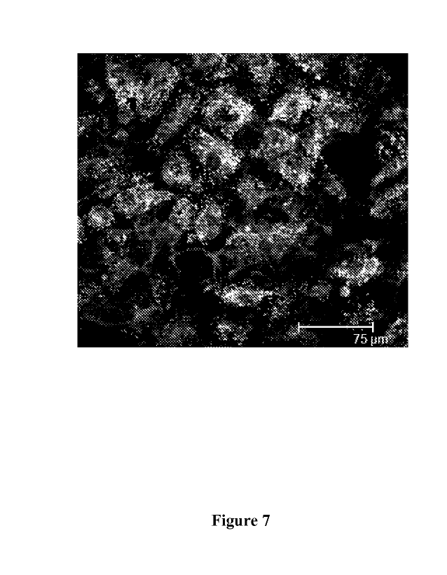

### FIG. 7:

FIG. 7 shows the image obtained in confocal microscopy (bar: 75 μm, objective ×40) of endothelial cells HUVECs adhering to the ePTFE substrate on which the PEI (PSS-PAH)₃ multilayer film was deposited, after 7 days of culture. The Von Willebrand factor is visualized by means of the fluorochrome Alexa Fluor 488 (λex: 494 nm, λem: 517 nm) and appears light grey. The dark grey circles that appear in the middle of the light grey parts represent the nuclei, which were labelled with propidium iodide (λex: 536 nm, λem: 617 nm).

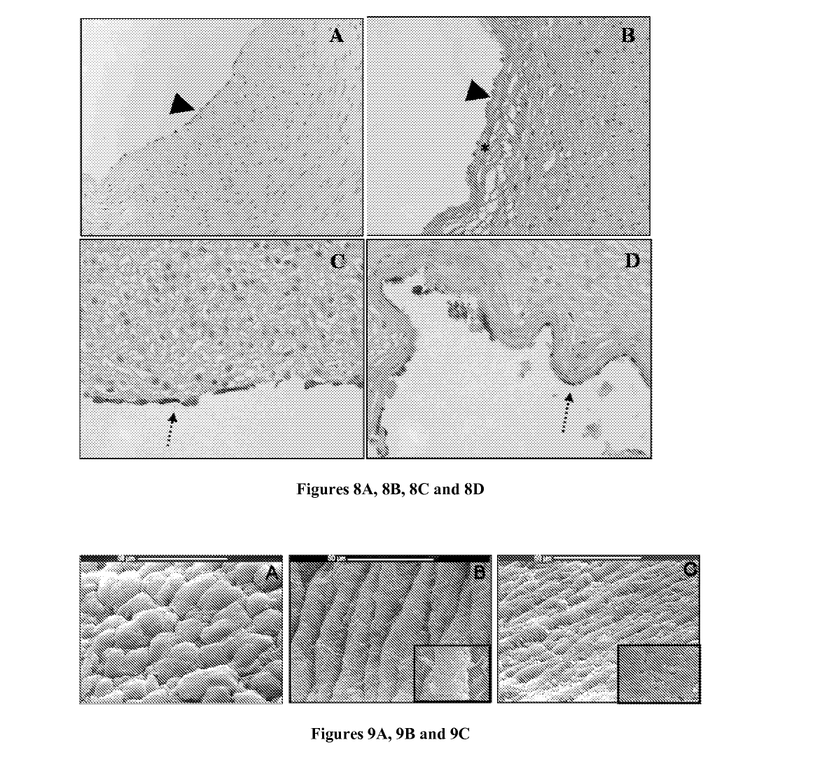

### FIGS. 8A, 8B, 8C, 8D:

FIG. 8A shows the image of a histological section of a re-endothelialized artery on which no polyelectrolyte multilayer film was deposited. Basic staining is with haematoxylin-eosin-Safran. The magnification is 20.

FIG. 8B shows the image of a histological section of a re-endothelialized artery on which a (PAH-PSS)₃-PAH polyelectrolyte multilayer film was deposited. Basic staining is with haematoxylin-eosin-Safran. The magnification is 20.

FIG. 8C shows the image obtained in immunohistochemistry, revealing the PECAM-1 membrane receptor expressed on the surface of the endothelial cells sown in the lumen of the artery, on which no polyelectrolyte multilayer film was deposited. It is revealed with a peroxidase, and the counter-staining is carried out with haematoxylin. The magnification is 20.

FIG. 8D shows the image obtained in immunohistochemistry, revealing the PECAM-1 membrane receptor expressed on the surface of the endothelial cells sown in the lumen of the artery on which a (PAH-PSS)₃-PAH polyelectrolyte multilayer film was deposited. It is revealed with a peroxidase, and the counter-staining is carried out with haematoxylin. The magnification is 20.

### FIGS. 9A, 9B, 9C:

FIG. 9A shows an image obtained after observation with the scanning electron microscope (bar: 50 μm), of endothelialized umbilical arteries on which no polyelectrolyte multilayer film was deposited.

FIG. 9B shows an image obtained after observation with the scanning electron microscope (bar: 50 μm), of endothelialized umbilical arteries on which a (PAH-PSS)₃-PAH polyelectrolyte multilayer film was deposited.

FIG. 9C shows an image obtained after observation with the scanning electron microscope (bar: 50 μm), of a fresh artery (control).

### FIGS. 10A, 10B, 10C, 10D:

FIG. 10A shows the image obtained after observation in confocal laser scanning microscopy (objective 40) after PECAM-1 labelling, of endothelialized arteries on which no polyelectrolyte multilayer film was deposited, in static conditions after one week of culture.

FIG. 10B shows the image obtained after observation in confocal laser scanning microscopy (objective 40) after PECAM-1 labelling, of endothelialized arteries on which no polyelectrolyte multilayer film was deposited, in dynamic conditions (endothelialized arteries subjected to a shearing stress of 1 Pa for 1 hour) after one week of culture.

FIG. 10C shows the image obtained after observation in confocal laser scanning microscopy (objective 40) after PECAM-1 labelling, of endothelialized arteries on which a (PAH-PSS)₃-PAH polyelectrolyte multilayer film was deposited, in static conditions after one week of culture.

FIG. 10D shows the image obtained after observation in confocal laser scanning microscopy (objective 40) after PECAM-1 labelling, of endothelialized arteries on which a (PAH-PSS)₃-PAH polyelectrolyte multilayer film was deposited, in dynamic conditions (endothelialized arteries subjected to a shearing stress of 1 Pa for 1 hour) after one week of culture.

### FIGS. 11A, 11B, 11C, 11D:

FIG. 11A shows the image obtained after observation with the scanning electron microscope (bar: 50 μm) after PECAM-1 labelling, of endothelialized arteries on which no polyelectrolyte multilayer film was deposited, in static conditions after one week of culture.

FIG. 11B shows the image obtained after observation with the scanning electron microscope (bar: 50 μm) after PECAM-1 labelling, of endothelialized arteries on which no polyelectrolyte multilayer film was deposited, in dynamic conditions (endothelialized arteries subjected to a shearing stress of 1 Pa for 1 hour) after one week of culture.

FIG. 11C shows the image obtained after observation with the scanning electron microscope (bar: 50 μm) after PECAM-1 labelling, of endothelialized arteries on which a (PAH-PSS)₃-PAH polyelectrolyte multilayer film was deposited, in static conditions after one week of culture.

FIG. 11D shows the image obtained after observation with the scanning electron microscope (bar: 50 μm) after PECAM-1 labelling, of endothelialized arteries on which a (PAH-PSS)₃-PAH polyelectrolyte multilayer film was deposited, in dynamic conditions (endothelialized arteries subjected to a shearing stress of 1 Pa for 1 hour) after one week of culture.

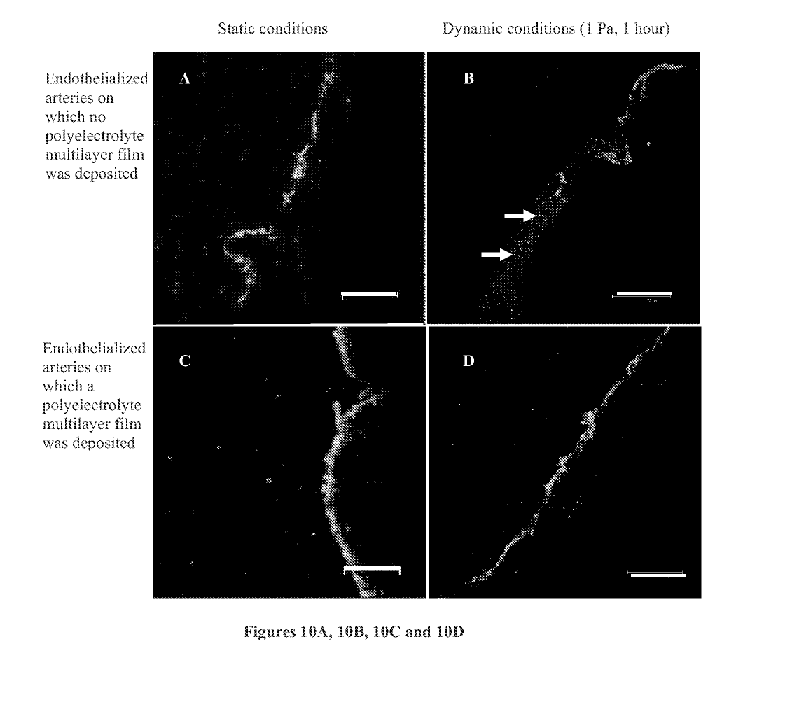

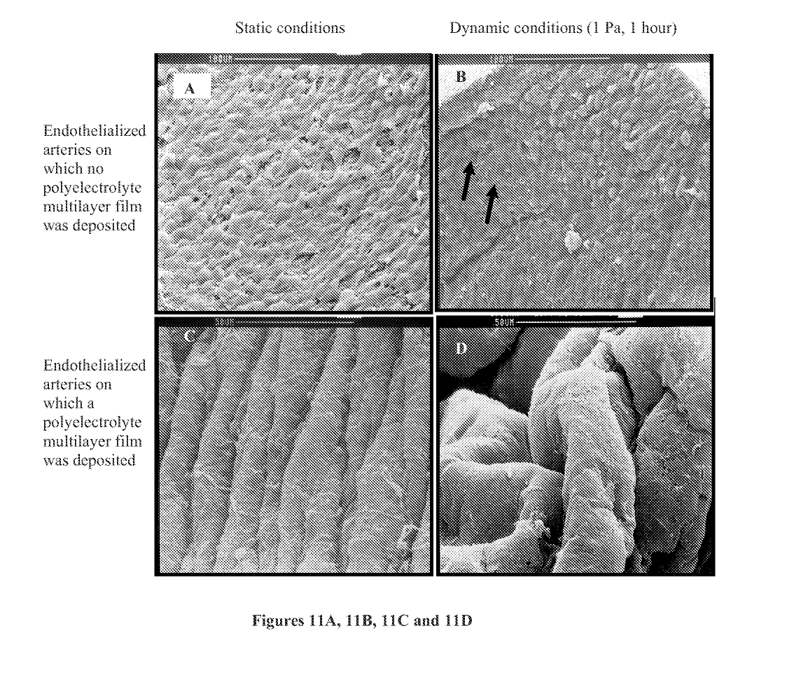

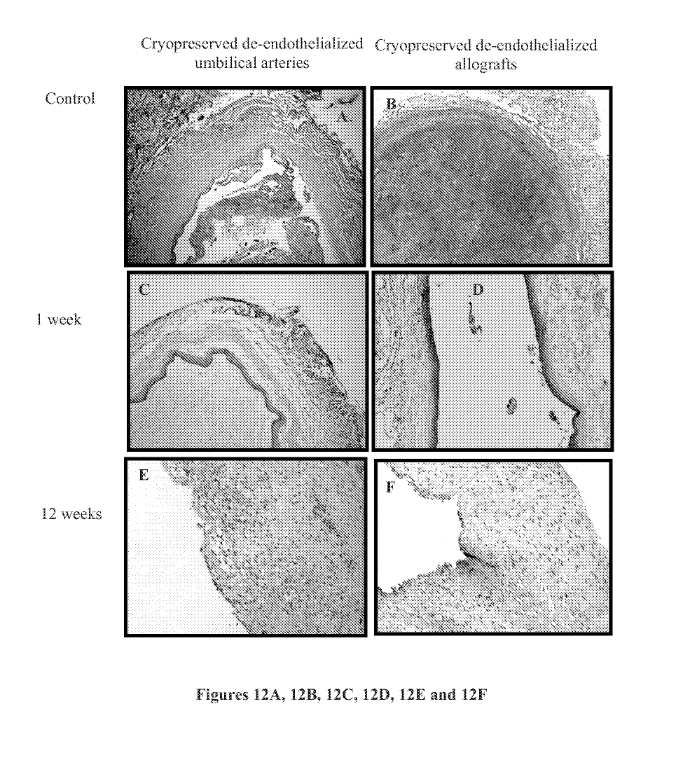

### FIGS. 12A, 12B, 12C, 12D, 12E, 12F:

FIG. 12A shows an image obtained after observation of a histological section of cryopreserved de-endothelialized rabbit umbilical arteries on which no polyelectrolyte multilayer film was deposited, one week after implantation (control).

FIG. 12B shows an image obtained after observation of a histological section of de-endothelialized rabbit allografts on which no polyelectrolyte multilayer film was deposited, one week after implantation (control).

FIG. 12C shows an image obtained after observation of a histological section of cryopreserved de-endothelialized rabbit umbilical arteries on which a (PAH-PSS)₃ polyelectrolyte multilayer film was deposited, one week after implantation.

FIG. 12D shows an image obtained after observation of a histological section of de-endothelialized rabbit allografts on which a (PAH-PSS)₃ polyelectrolyte multilayer film was deposited, one week after implantation.

FIG. 12E shows an image obtained after observation of a histological section of cryopreserved de-endothelialized rabbit umbilical arteries on which a (PAH-PSS)₃ polyelectrolyte multilayer film was deposited, 12 weeks after implantation.

FIG. 12F shows an image obtained after observation of a histological section of de-endothelialized rabbit allografts on which a (PAH-PSS)₃ polyelectrolyte multilayer film was deposited, 12 weeks after implantation.

In FIGS. 12A to 12F, basic staining was carried out with haematoxylin-eosin-Safran and the magnification is 20.

### FIG. 13A, 13B, 13C, 13D, 13E, 13F:

FIG. 13A shows an image obtained after observation with the scanning electron microscope (bar: 1 mm) of cryopreserved de-endothelialized rabbit umbilical arteries on which no polyelectrolyte multilayer film was deposited, one week after implantation (control).

FIG. 13B shows an image obtained after observation with the scanning electron microscope (bar: 1 mm) of de-endothelialized rabbit allografts on which no polyelectrolyte multilayer film was deposited, one week after implantation (control).

FIG. 13C shows an image obtained after observation with the scanning electron microscope (bar: 1 mm) of cryopreserved de-endothelialized rabbit umbilical arteries on which a (PAH-PSS)₃ polyelectrolyte multilayer film was deposited, one week after implantation.

FIG. 13D shows an image obtained after observation with the scanning electron microscope (bar: 1 mm) of de-endothelialized rabbit allografts on which a (PAH-PSS)₃ polyelectrolyte multilayer film was deposited, one week after implantation.

FIG. 13E shows an image obtained after observation with the scanning electron microscope (bar: 1 mm) of cryopreserved de-endothelialized rabbit umbilical arteries on which a (PAH-PSS)₃ polyelectrolyte multilayer film was deposited, 12 weeks after implantation.

FIG. 13F shows an image obtained after observation with the scanning electron microscope (bar: 1 mm) of de-endothelialized rabbit allografts on which a (PAH-PSS)₃ polyelectrolyte multilayer film was deposited, 12 weeks after implantation.

### FIGS. 14A, 14B, 14C:

FIG. 14A shows an image obtained after echo-Doppler observation 10 weeks after implantation for the control carotid, which is the native rabbit carotid (control). The tracing at the bottom of the image shows that the velocity of the blood is 40 cm/s.

FIG. 14B shows an image obtained after echo-Doppler observation 10 weeks after implantation for cryopreserved de-endothelialized umbilical arteries on which a (PAH-PSS)₃ polyelectrolyte multilayer film was deposited. The tracing at the bottom of the image shows that the velocity of the blood is 40 cm/s.

FIG. 14C shows an image obtained after echo-Doppler observation 10 weeks after implantation for cryopreserved de-endothelialized umbilical arteries on which no polyelectrolyte multilayer film was deposited. The tracing at the bottom of the image shows that the velocity of the blood is zero: the blood is not circulating, as the artery is blocked.

### FIGS. 15A, 15B, 15C, 15D, 15E:

FIG. 15A shows the image obtained by observation in phase-contrast microscopy (Objective 20) of a glass slide covered with fibronectin and then with endothelial progenitors, at 4 days of culture.

FIG. 15B shows the image obtained by observation in phase-contrast microscopy (Objective 20) of a glass slide on which a (PAH-PSS)₃-PAH polyelectrolyte multilayer film was deposited and then endothelial progenitors were sown, at 4 days of culture.

FIG. 15C shows the image obtained by observation in phase-contrast microscopy (Objective 20) of a glass slide covered with fibronectin and then with endothelial progenitors, at 14 days of culture.

FIG. 15D shows the image obtained by observation in phase-contrast microscopy (Objective 20) of a glass slide on which a (PAH-PSS)₃-PAH polyelectrolyte multilayer film was deposited and then endothelial progenitors were sown, at 14 days of culture.

FIG. 15E shows the image obtained by observation in phase-contrast microscopy (Objective 20) of TCPS (“treated cell culture” polystyrene) covered with a monolayer of mature endothelial cells obtained from the rabbit jugular vein (JVEC) (control).

### FIGS. 16A, 16B, 16C, 16D, 16E, 16F, 16G, 16H, 16I, 16J, 16K, 16L:

FIG. 16A shows the image obtained after observation in confocal laser scanning microscopy after 14 days of culture (Objective 40) of jugular vein endothelial cells (control) whose PECAM-1 membrane receptor had been labelled. The labelling is indirect immunolabelling: a primary antibody which recognizes the PECAM-1 antigen is recognized by a secondary antibody labelled with a fluorochrome (Alexa® 488). The adhesion and spread of the cells on the substrate were evaluated from the appearance of actin fibres. PECAM-1 revealed by a fluorochrome (Alexa® 488) appears light grey.

FIG. 16B shows the image obtained after observation in confocal laser scanning microscopy after 14 days of culture (Objective 40) of jugular vein endothelial cells (control) whose intracellular marker (von Willebrand factor (vWF)) had been labelled. The labelling is indirect immunolabelling: a primary antibody which recognizes the vWF antigen is recognized by a secondary antibody labelled with a fluorochrome (Alexa® 488). The adhesion and spread of the cells on the substrate were evaluated from the appearance of actin fibres. The intracellular marker (von Willebrand factor (vWF)) revealed by a fluorochrome (Alexa® 488) appears light grey.

FIG. 16C shows the image obtained after observation in confocal laser scanning microscopy after 14 days of culture (Objective 40) of jugular vein endothelial cells (control) whose cytoskeleton is revealed by recognition by an antibody bound to a fluorochrome (Alexa® 488). The adhesion and spread of the cells on the substrate were evaluated from the appearance of actin fibres. The cytoskeleton appears light grey.

FIG. 16D shows the image obtained after observation in confocal laser scanning microscopy after 14 days of culture (Objective 40) of jugular vein endothelial cells (control) whose LDL had been coupled to Dil (fluorescent molecule). The cells' capacity for incorporating LDLs is a characteristic of the functionality of mature endothelial cells. The LDLs coupled to Dil (fluorescent molecule) appear grey. Syto 16 (marker specific to the nucleus) appears light grey, making it possible to show the perinuclear distribution of the LDLs coupled to Dil.

FIG. 16E shows the image obtained after observation in confocal laser scanning microscopy after 14 days of culture (Objective 40) of EPC cells sown on a (PAH-PSS)₃-PAH polyelectrolyte multilayer film and whose PECAM-1 membrane receptor had been labelled by the same method as for FIG. 16A. The adhesion and spread of the cells on the substrate were evaluated from the appearance of actin fibres. PECAM-1 revealed by a fluorochrome (Alexa® 488) appears light grey.

FIG. 16F shows the image obtained after observation in confocal laser scanning microscopy after 14 days of culture (Objective 40) of EPC cells sown on a (PAH-PSS)₃-PAH polyelectrolyte multilayer film, whose intracellular marker (von Willebrand factor (vWF)) was labelled by the same method as for FIG. 16B. The adhesion and spread of the cells on the substrate were evaluated from the appearance of actin fibres. The intracellular marker (von Willebrand factor (vWF)) revealed by a fluorochrome (Alexa® 488) appears light grey.

FIG. 16G shows the image obtained after observation in confocal laser scanning microscopy after 14 days of culture (Objective 40) of EPC cells sown on a (PAH-PSS)₃-PAH polyelectrolyte multilayer film, whose cytoskeleton is revealed by recognition by an antibody bound to a fluorochrome (Alexa® 488). The adhesion and spread of the cells on the substrate were evaluated from the appearance of actin fibres. The cytoskeleton appears light grey.

FIG. 16H shows the image obtained after observation in confocal laser scanning microscopy after 14 days of culture (Objective 40) of EPC cells sown on a (PAH-PSS)₃-PAH polyelectrolyte multilayer film, whose LDL had been coupled to Dil (fluorescent molecule). The cells' capacity for incorporating LDLs is a characteristic of the functionality of mature endothelial cells. The LDLs coupled to Dil (fluorescent molecule) appear grey. Syto 16 (marker specific to the nucleus) appears light grey, making it possible to show the perinuclear distribution of the LDLs coupled to Dil.

FIG. 16I shows the image obtained after observation in confocal laser scanning microscopy after 14 days of culture (Objective 40) of EPC cells sown on a layer of fibronectin, whose PECAM-1 membrane receptor had been labelled by the same method as for FIG. 16A. The adhesion and spread of the cells on the substrate were evaluated from the appearance of actin fibres. PECAM-1 revealed by a fluorochrome (Alexa® 488) appears light grey.

FIG. 16J shows the image obtained after observation in confocal laser scanning microscopy after 14 days of culture (Objective 40) of EPC cells sown on a layer of fibronectin, whose intracellular marker (von Willebrand factor (vWF)) had been labelled by the same method as for FIG. 16B. The adhesion and spread of the cells on the substrate were evaluated from the appearance of actin fibres. The intracellular marker (von Willebrand factor (vWF)) revealed by a fluorochrome (Alexa® 488) appears light grey.

FIG. 16K shows the image obtained after observation in confocal laser scanning microscopy after 14 days of culture (Objective 40) of EPC cells sown on a layer of fibronectin, whose cytoskeleton is revealed by recognition by an antibody bound to a fluorochrome (Alexa® 488). The adhesion and spread of the cells on the substrate were evaluated from the appearance of actin fibres. The cytoskeleton appears light grey.

FIG. 16L shows the image obtained after observation in confocal laser scanning microscopy after 14 days of culture (Objective 40) of EPC cells sown on a layer of fibronectin, whose LDL had been coupled to Dil (fluorescent molecule). The cells' capacity for incorporating the LDLs is a characteristic of the functionality of mature endothelial cells. The LDLs coupled to Dil (fluorescent molecule) appear grey. Syto 16 (marker specific to the nucleus) appears light grey, making it possible to show the perinuclear distribution of the LDLs coupled to Dil.

### FIGS. 17A, 17B, 17C:

FIG. 17A presents a graph that corresponds to semiquantitative investigation of the fluorescence in FIGS. 16A, 16E and 16I for the PECAM-1 membrane receptor. The grey level per pixel is shown on the ordinate. The origin of the endothelial cells is shown on the abscissa:
jugular vein endothelial cells (JVE) (fluorescence in FIG. 16A),
EPC cells sown on a layer of fibronectin after 14 days of culture (Fn or F) (fluorescence in FIG. 16E) and,
EPC cells sown on a polyelectrolyte multilayer film after 14 days of culture (PEM) (fluorescence in FIG. 16I).

FIG. 17B presents a graph that corresponds to semiquantitative investigation of the fluorescence in FIGS. 16B, 16F and 16J for the intracellular marker vWF. The grey level per pixel is shown on the ordinate. The origin of the endothelial cells is shown on the abscissa:
jugular vein endothelial cells (JVE) (fluorescence in FIG. 16B),
EPC cells sown on a layer of fibronectin after 14 days of culture (Fn or F) (fluorescence in FIG. 16F) and,
EPC cells sown on a polyelectrolyte multilayer film after 14 days of culture (PEM) (fluorescence in FIG. 16J).

FIG. 17C presents a graph that corresponds to semiquantitative investigation of the fluorescence in FIGS. 16A, 16E and 16I for the LDL coupled to Di with Sito 16. The grey level per pixel is shown on the ordinate. The origin of the endothelial cells is shown on the abscissa:
jugular vein endothelial cells (JVE) (fluorescence in FIG. 16D),
EPC cells sown on a layer of fibronectin after 14 days of culture (Fn or F) (fluorescence in FIG. 16H) and,
EPC cells sown on a polyelectrolyte multilayer film after 14 days of culture (PEM) (fluorescence in FIG. 16L).

In FIGS. 17A to 17C, the 3-star symbol *** denotes that the fluorescence of the EPC cells sown on a layer of fibronectin is significantly different from that of the jugular vein endothelial cells with an error probability less than 0.001%.

### FIG. 18:

FIG. 18 shows the result of the viability test on endothelial cells by assay with Alamar Blue®.
ΔOD=[OD(570 nm)_{exp.}−OD(630 nm)_{exp.}]−[OD(570 nm)_{cont.}−OD(630 nm)_{cont.}] with exp.=experimental, cont.=control without cells and Δ=difference, is shown on the ordinate.
The origin of the endothelial cells is shown on the abscissa: jugular vein endothelial cells (JVE), EPC cells sown on a layer of fibronectin after 14 days of culture (F) and EPC cells sown on a polyelectrolyte multilayer film after 14 days of culture (PEM).
The 2-star symbol ** denotes that the difference in absorbance of the EPC cells sown on a layer of fibronectin is significantly different from that of the jugular vein endothelial cells with an error probability less than 0.05%.

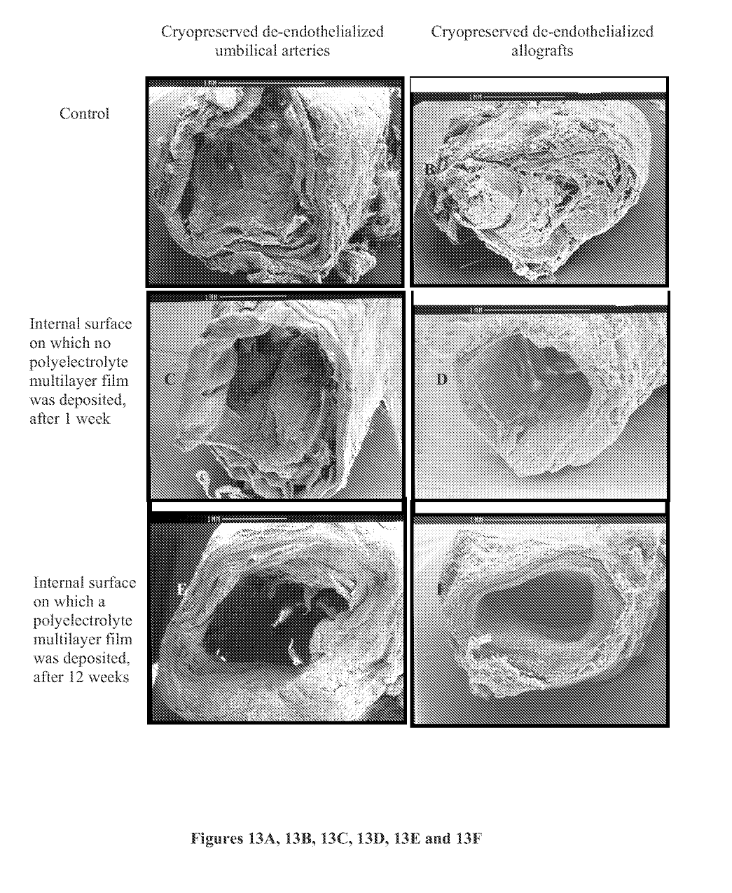

### FIG. 19:

FIG. 19 is a schematic diagram of the shearing chamber used during investigation of differentiation of EPCs sown on an artery, on which a (PAH-PSS)₃-PAH polyelectrolyte multilayer film had been, or had not been, deposited.

### FIG. 20:

FIG. 20 shows the calibration curve of the peristaltic pump.
The shear stress in pascal is shown on the ordinate.
The graduation is shown on the abscissa.

### EXAMPLES

### Example 1

### Preparation of Polyelectrolyte Multilayer Films Deposited on a Substrate

### 1.1. Preparation of the Substrates

### 1.1.1. Preparation of the Glass Slides

The glass slides are washed to reveal the silica (Si-) and to make the surface of the slides negative.

More precisely, the glass slides are washed for 15 min at 100° C. in a 0.01 M solution of sodium dodecyl sulphate (SDS). Three washings are then carried out with filtered distilled water. The slides are then immersed in 0.12 M hydrochloric acid solution for 15 min at 100° C. Three washings are carried out with filtered distilled water. The slides are stored at 4° C. in filtered distilled water before treatment.

1.1.2. Preparation of the ePTFE

Patches of expanded polytetrafluoroethylene ePTFE with diameter of 9 mm are prepared from tubular vascular prostheses of ePTFE (6 mm inside diameter and fibril length 25 μm). These patches are then glued in 48-well culture plates. The polyelectrolyte multilayer films are then constructed directly on the ePTFE inside the wells. Preliminary studies showed absence of cytotoxicity of the glue.

### 1.1.3. Composition of the Complete Medium

### Composition of the Culture Medium for the Cells

Human AB serum (obtained from healthy volunteer donors) used at 20%. It is decomplemented at 56° C. for 30 min.
M199 and RPMI 1640 v/v (Gibco BRL, France).
2 mM of glutamine (Gibco BRL, France).
100 U/mL of penicillin (Gibco BRL, France).
100 μg/mL of streptomycin (Gibco BRL, France).
2.5 μg/mL of Fungizone® (Gibco BRL, France).
20 mM HEPES (Sigma, France).

When RPMI 1640/M199 mixture is supplemented with these additives, it forms the so-called “complete” medium. The shelf life of the complete medium, stored at 4° C., does not exceed 2 weeks.

### 1.1.4. Preparation of the Cryopreserved, De-Endothelialized Arteries

The arteries are recovered from the human umbilical cord. Using two surgical forceps, the umbilical cord is dilacerated and lengths of arteries of at least 6 cm are isolated and immersed in buffer (Hank's Balanced Salt Solution HBSS). After rinsing several times, generally three to five (until the artery no longer contains blood) the arteries are put in cryotubes containing 1 mL of a freezing solution, which is constituted of 70% of complete medium supplemented with 10% of dimethylsulphoxide (DMSO, Sigma, France) and 20% of fetal calf serum (Gibco BRL, France), previously decomplemented at 56° C. for 30 min.

The cryotubes are stored overnight at −80° C., and then immersed in liquid nitrogen at −180° C. The shelf life is normally 6 months (the time required for carrying out serological tests when using allografts taken from cadavers).

The umbilical arteries are thawed by immersing the cryotubes in a water bath at 37° C. They are then washed with a decontaminating solution, which is constituted of RPMI 1640 medium (Gibco BRL, France) supplemented with 100 IU/mL of penicillin (Gibco BRL, France), 100 μg/mL of streptomycin (Gibco BRL, France) and 2.5 μg/mL of Fungizone® (Gibco BRL, France).

The lumen of the artery is washed three times with buffer (HBSS), and then it is filled with a digesting solution (trypsin/EDTA 0.25%). After incubation at 37° C. for 20 min, the artery is washed with 2 mL of medium containing whole serum. The arteries called “de-endothelialized arteries” hereinafter are those that have undergone this process of cryopreservation.

### 1.2. Preparation of the Solutions of Polyelectrolytes: PAH, PSS, PEI

The polyelectrolyte multilayer films are constituted of alternating solutions of polycations and polyanions.

### 1.2.1. Materials Used

### Buffer: Solution of Tris/NaCl (Tris 10 mM and NaCl 150 mM).

### Polyelectrolytes Used:

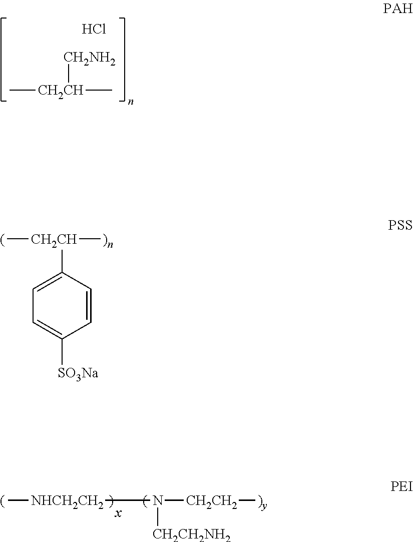

### Chemical Structure of the Polyelectrolytes Used for Constructing the Polyelectrolyte Multilayer Films

### Polycations Used:

Poly(allylamine hydrochloride) (PAH) (Sigma Aldrich, France, MW=70 kDa) 5 g/L in buffer (for arteries and glass) or in 1M NaCl (ePTFE).
Polyethyleneimine (PEI) 10 g/L (Sigma Aldrich, France, MW=750 kDa) in 1M NaCl solution.

### Polyanion Used:

Poly(sodium-4-styrene sulphonate) (PSS) (Sigma Aldrich, France, MW=70 kDa) 5g/L in buffer (for arteries and glass) or in 1M NaCl (ePTFE).

The use of these polyelectrolytes is described in the following works:
C. Boura, P. Menu, E. Payan, J. C. Voegel, S. Muller, J. F. Stoltz, Biomaterials, “Endothelial cells grown on multilayered thin polyelectrolyte films: An evaluation of a new versatile surface modification” 24, 3521-3530, 2003.
C. Boura, S. Muller, D. Vautier, D. Dumas, P. Schaaf, J-C Voegel, J-F Stoltz, P. Menu, Biomaterials “Endothelial cell—interactions with polyelectrolyte multilayer films” 26, 4568-75, 2005.
D. Vautier, V. Karsten, C. Egles, J. Chluba, P. Schaaf, J. C. Voegel, J. Ogier. J Biomater Sci Polym Ed. “Polyelectrolyte multilayer films modulate cytoskeletal organization in chondrosarcoma cell” 13(6), 713-32, 2002.
P. Tryoen-Tóth, D. Vautier, Y. Haikel, J. C. Voegel, P. Schaaf, J. Chluba, J. Ogier, J Biomed Mater Res. “Viability, adhesion, and bone phenotype of osteoblast-like cells on polyelectrolyte multilayer films” 60(4), 657-67, 2002.
   1.2.2. Construction of (PAH-PSS)₃, (PAH-PSS)₃-PAH and PEI-(PSS-PAH)₃ Polyelectrolyte Multilayer Films on Substrates (Glass, ePTFE or Artery)

### Construction of Polyelectrolyte Multilayer Films

The polyelectrolyte multilayer films were deposited in the lumen of the previously de-endothelialized umbilical arteries, on glass slides or on ePTFE, as appropriate. Assembly is carried out at room temperature by successive depositions of the substrate alternately in a solution of polycation and of polyanion. After washing twice with Tris/NaCl buffer for the glass and arteries as substrates, and with distilled water for the ePTFE substrate, the substrates are brought in contact with
the solution of polycations (PAH) for 15 to 30 min, at room temperature, for constructing the (PAH-PSS)₃ and (PAH-PSS)₃-PAH multilayer films (for the case when the substrate is glass or an artery), or,
the solution of polycations (PEI) for constructing the PEI-(PSS-PAH)₃ multilayer film (when the substrate is ePTFE).
This stage is followed by three washings with Tris/NaCl buffer for the glass and arteries as substrates, and with distilled water for the ePTFE substrate, for removing the free polyelectrolytes. The procedure is repeated with a solution of polyanion (PSS), then a solution of polycation (PAH). A time of between 8 and 20 min is necessary to allow the solutions of PSS and PAH to be adsorbed alternately on the substrate. Between each deposition, three washings, with Tris/NaCl buffer for the glass and arteries as substrates, and with distilled water for the ePTFE substrate, are carried out. The (PAH-PSS)₃ (ending with a negative charge), (PAH-PSS)₃-PAH and PEI-(PSS-PAH)₃ (ending with a positive charge) polyelectrolyte multilayer films are constructed progressively. All the stages of construction are carried out while keeping the substrate in a solution of polyelectrolytes or of distilled water to prevent the surface drying out.

### Preservation of the Substrates on Which a Polyelectrolyte Multilayer Film Has Been Deposited and Definition of the Substrates Used as Control for the Subsequent Experiments

### * In the Case of Glass Slides on Which PAH-PSS-PAH)₃ Has Been Deposited

Before each experiment, the cell culture plates containing the glass slides are exposed to UV for 15 min for sterilization.

Glass slides covered with fibronectin (Sigma, France) at a concentration of less than 250 μg/mL are used as positive controls.

* In the Case of ePTFE on Which PEI-(PSS-PAH)₃ Has Been Deposited

The ePTFE substrate on which a PEI-(PSS-PAH)₃ polyelectrolyte multilayer film has been deposited is dried at least overnight at 4° C. after deposition of the multilayer film and prior to use. It is stored for at most 15 days at 4° C.

The TCPS substrate (Tissue Culture Polystyrene Surface) is the material most commonly used for cell culture, and it is a polymer that is widely used for studying the mechanisms of interactions between cells and artificial material. It is used as a positive control.

### * In the Case of Arteries on Which (PAH-PSS)₃ or PAH-PSS)₃-PAH Have Been Deposited

The de-endothelialized arteries on which no polyelectrolyte multilayer film had been deposited, are submitted to several injections of washing buffer and are regarded as controls (control artery). The arteries on which polyelectrolyte multilayer films had been deposited and the control arteries are stored overnight at 4° C. in a decontaminating solution before use. The latter is constituted of RPMI 1640 medium (Gibco BRL, France) supplemented with 100 IU/mL of penicillin (Gibco BRL, France), 100 μg/mL of streptomycin (Gibco BRL, France) and 2.5 μg/mL of Fungizone® (Gibco BRL, France).

### 1.3. Validation of the Deposition of the Polyelectrolyte Multilayer Film

1.3.1. When the Substrate is ePTFE

Verification of uniform deposition of the polyelectrolyte multilayer film on the ePTFE substrate was carried out by confocal laser scanning microscopy. FIG. 1 shows demonstration of covering of the entire surface with the polyelectrolyte multilayer film [PEI-(PSS-PAH)₂-PSS-PAH*] by using the polycation poly(allylamine) hydrochloride coupled covalently to rhodamine (PAH*) (λexcitation=541 nm, λemission.=572 nm, ICS, UPR 22 CNRS, Strasbourg, France) during construction of the polyelectrolyte multilayer films.

### 1.3.2. When the Substrate is a Vessel or an Artery

Verification of uniform deposition of the polyelectrolyte multilayer film on the internal surface of vessels was carried out by confocal laser scanning microscopy. FIGS. 2A to 2E show demonstration of covering of the entire internal surface of an artery with the polyelectrolyte multilayer film [(PAH-PSS)₂-PAH*-PSS-PAH*] by using the polycation poly(allylamine) hydrochloride coupled to rhodamine (PAH*) during construction of the polyelectrolyte multilayer films.

### Example 2

### Mechanical Evaluation of the Arterial Matrix

### 2.1. Description of the Test Bench and of the Experiment

The mechanical tests are carried out by means of a test bench developed in the laboratory. The pressure is supplied by a pressure detector (XTC-190M-0.35 BARVG, Kulite, Inc) located at pump outlet (EX303C-50, Prodera, France). The information is representative of the pressure exerted on the inside walls of the artery. The outside diameter of the artery is evaluated by a CCD camera (FZS 1024, Sensopart UK Ltd), which measures its deformation. The CCD unit delivers a voltage in relation to the amount of light received by a neon lamp, which serves as the standard light source.

Each end of the artery (treated and control) is mounted in plastic tips, then the artery is fixed in a plexiglas chamber filled with physiological saline solution preheated to 37° C. The artery must be kept taut. Using a syringe fitted with a tube, the interior of the artery is filled with physiological saline solution, avoiding the formation of air bubbles. The free end of the artery is clamped to close the circuit. The pump thus increases the pressure in this closed circuit.

The parameters are entered in software for controlling the pump. The pressure in the artery increases by constant steps every 15 seconds up to 230 mmHg (with increments of 30 mmHg). The outside diameter of the artery is recorded for each pressure.

The percentage deformation is calculated according to the following equation:

ΔD=100×(Dp−DO)/DO
in which
Dp is the diameter corresponding to each pressure.
DO is the diameter at 0 mmHg

The elasticity of the arteries corresponds to the straight line ΔD over pressure, measured at physiological pressures (between 80 and 150 mmHg).

### 2.2. Results

FIGS. 3 and 4 show that the mechanical properties of the arterial wall after deposition of the (PAH-PSS)₃-PAH polyelectrolyte multilayer film on a de-endothelialized artery are restored. In fact, the percentage deformation of the artery as a function of the pressure exerted on said artery is similar for fresh arteries (•) and de-endothelialized arteries on which a (PAH-PSS)₃-PAH polyelectrolyte multilayer film has been deposited (▴), and is greater than that of the de-endothelialized arteries on which no polyelectrolyte multilayer film was deposited (▪).

### Example 3

### Proliferation of Endothelial Cells on ePTFE on Which a PEI-(PSS-PAH)₃ Polyelectrolyte Multilayer Film Has Been Deposited

### 3.1. Culture of the Endothelial Cells

### 3.1.1. Composition of the Solutions Used

HBSS buffer (Hank's balanced salt solution) (Sigma, France) without Ca²⁺ or Mg²⁺ containing 0.4 g/L of KCl, 8 g/L of NaCl, 0.06 g/L of KH₂PO₄, 0.04778 g/L of Na₂HPO₄, 1 g/L of D-glucose, 0.011 g/L of phenol red, to 1 L of distilled water (pH 7.2). It must be filtered on 0.22 μm and stored at 4° C.
Trypsin-EDTA digesting solution at 0.25% (Sigma, France) containing 2.5 g of porcine trypsin and 0.2 g of EDTA-Na₄ in 100 mL of HBSS. Trypsin is a proteolytic enzyme that hydrolyses peptide bonds.
Culture medium or “complete medium” constituted of:
   M199 medium and RPMI 1640 medium in equal volumes (GibcoBRL, France)
   20% of decomplemented human serum AB (obtained from healthy volunteer donors).
   2 mM of glutamine (GibcoBRL, France)
   100 U/mL of penicillin (GibcoBRL, France)
   100 μg/mL of streptomycin (GibcoBRL, France)
   2.5 μg/mL of Fungizone (GibcoBRL, France)
   20 mM of HEPES (Sigma, France)
Phosphate-Buffered Saline (PBS) containing NaCl 137 mM, KCl 2.7 mM, Na₂HPO₄ 10 mM, KH₂PO₄ 1.4 mM.

### 3.1.2. Cells Used

The endothelial cells required for this study are obtained from human umbilical veins (HUVECs Human Umbilical Vein Endothelial Cells). They are taken from umbilical cords of neonates (donated by the Nancy District Maternity Hospital). The cords are obtained from healthy donors, after their consent. Collected immediately after delivery of the placenta, the cord is cut to a size of 20 to 25 cm and immediately put in a 75 cm² culture bottle containing 150 mL of sterile HBSS. Quickly cooled to 4° C., the cord is used as soon as possible. It can be kept for 4-6 hours.

### 3.1.3. Isolation of Endothelial Cells From Umbilical Cords

The cells are cultured according to Jaffe's method (E. A. Jaffe, R. L. Nachman, C. G. Becker, C. R. Minick, J Clin Invest. “Culture of human endothelial cells derived from umbilical veins. Identification by morphologic and immunologic criteria.” 52(11), 2745-56, 1973) in several stages:
3.1.3.1. Washing of the Umbilical Vein

The HBSS buffer is removed from the flask and the cord is placed in a sterile culture flask. The exterior of the cord is cleaned with 75% ethanol. A tap is fixed to one of the orifices of the umbilical vein and tied firmly to the cord. Using a syringe, the vein is washed three times with HBSS buffer (filtered and preheated to 37° C.) to remove the blood from it. Then the other end of the cord is clamped.
3.1.3.2. Detachment of the Cells From the Umbilical Vein

15-20 mL of the digesting solution (filtered and preheated to 37° C.) is injected into the vein until it is sufficiently dilated. The cord is immersed in 200 mL of HBSS; the whole is put in a water bath at 37° C. for 10 minutes. The cord is then gently placed in a culture flask and massaged for a few seconds. It is then unclamped above a 50 mL plastic tube (Polylabo, France) containing 20 mL of complete medium to stop the action of the trypsin, and in which the digesting solution containing the free endothelial cells is collected. The vein is then rinsed with HBSS buffer so that any cells still present are entrained into the tube. The cellular suspension is centrifuged at 1200 rpm (300 g) for 6 minutes, at room temperature. After centrifugation and deposition of the supernatant, the cellular pellet is resuspended in 10 mL of HBSS. Then a second centrifugation is carried out. The cells are resuspended in 5 mL of complete medium, sown in a 25 cm² culture bottle and put in an incubator at 37° C. (5% CO₂ and 95% air) and at saturation humidity.

### 3.1.4. Culture of the Cells

The day following extraction of the endothelial cells, the cells are washed twice with HBSS buffer, with small oscillating movements so as to remove the red blood cells. Then the cells are put back in the incubator with 5 mL of complete medium. The medium is renewed every other day. Normally, the cells are confluent after 5-7 days.

At confluence, the cells are washed twice with 5 mL of HBSS (preheated to 37° C.) and put in contact with 5 mL of trypsin-EDTA 0.125% (filtered), at 37° C., for 3 minutes.

The digesting action of the trypsin is stopped by adding 5 mL of complete medium. The cellular suspension is collected in sterile conical tubes and then centrifuged at 1200 rpm (300 g) for 6 minutes. The cells are then resuspended in 5 mL of complete medium.

3.1.5. Seeding of the Endothelial Cells on ePTFE on Which a Polyelectrolyte Multilayer Film Has Been Deposited

The cells are sown, after their second passage (P2), at a cell density of 5.10⁴ cells/well on ePTFE on which the PEI-(PSS-PAH)₃ polyelectrolyte multilayer film was deposited, on ePTFE on which a PAH monolayer was deposited, on ePTFE alone and on TCPS (Tissue Culture Polystyrene Surface) (positive control). The medium is changed every 3 days.

### 3.2. Evaluation of the Biocompatibility of the Surfaces

3.2.1 Evaluation of Cell Viability with Alamar blue®

Seifalian et al. (A. M. Seifalian, H. J. Salacinski, G. Punshon, B. Krijgsman, G. Hamilton, J. Biomed. Mater. Res. “A new technique for measuring the cell growth and metabolism of endothelial cells seeded on vascular prostheses.” 15, 55(4), 637-44, 2001) demonstrated that Alamar blue (Serotec Ltd, Kidlington, England) is an agent that has many advantages in evaluation of the metabolism of endothelial cells and therefore of the viability of the cells growing on vascular prostheses.

Alamar blue® redox assay (ABRA) (Alamar blue test) is a technique that has been used for monitoring the viability of endothelial cells seeded on vascular substitutes (ePTFE). With this technique, cellular proliferation, cytotoxicity and viability can be measured quantitatively. Alamar blue is composed of a redox indicator (colorimetric indicator), which changes colour in relation to chemical reduction of the culture medium. Alamar blue is reduced by mitochondrial activity, which is representative of cellular metabolic activity and therefore of cell viability.

Alamar Blue has interesting properties as it is soluble in the medium, stable in solution, nontoxic to the cells and produces changes that can be measured easily. The test does not require lysis of the cells, which makes it possible to follow the kinetics of the signal.

Measurement of cell viability is therefore based on the degree of oxidoreduction of Alamar blue determined by the difference between densitometric measurement at 570 nm (absorbance of the reduced compound) and at 630 nm (absorbance of the oxidized compound). Taking into account the partial overlap of the absorption spectra of the reduced compound (red) and of the oxidized compound (blue), the absorbance is measured at two wavelengths and the difference in optical density (OD) is determined according to the formula:

ΔOD=[OD(570nm)_{exp.}−OD(630nm)_{exp.}]−[OD(570nm)_{cont.}−OD(630nm)_{cont.}]

exp.=experimental; cont.=control without cells; Δ=difference

The procedure is as follows. The Alamar blue test is carried out according to the chosen protocol. The endothelial cells are sown on the surfaces for 1, 3, or 7 days. At the chosen time, the culture medium is replaced with fresh medium without serum containing 10% v/v of Alamar blue (the sensitivity of the Alamar blue technique depends on the volume ratio between Alamar blue and the DMEM medium (Dulbecco's Modified Eagle Medium) without phenol red (GibcoBRL, France)). 500 μL of this mixture is put in each well. The culture plate is put in the incubator at 37° C.

Densitometric measurement is carried out 3 hours after adding the marker. The difference in optical density (indicator of cell viability) is then determined Wells without cells are used as reference.

FIG. 5 shows the result of the viability test on endothelial cells sown on:
TCPS,
ePTFE,
ePTFE on which the PAH polyelectrolyte (monolayer) was deposited, or,
ePTFE on which the PEI-(PSS-PAH)₃ polyelectrolyte multilayer film was deposited.

After culture for one day, no difference in metabolic activity can be detected.

After culture for 3 days, a significant increase in metabolic activity of the cells is measured on TCPS. On the ePTFE substrates, the metabolic activity remains similar to that observed after culture for one day.

After culture for 7 days, the values of metabolic activity observed for the ePTFE on which the PEI-(PSS-PAH)₃ polyelectrolyte multilayer film was deposited (0.59±0.20) are similar to those observed for the TCPS substrate. However, for the same culture time, the values of metabolic activity observed for the ePTFE on which the PAH polyelectrolyte was deposited and for the ePTFE alone are significantly less than those observed for the ePTFE on which the PEI-(PSS-PAH)₃ polyelectrolyte multilayer film was deposited.

The endothelial cells therefore began to proliferate on the ePTFE on which the PEI-(PSS-PAH)₃ polyelectrolyte multilayer film was deposited after culture for three days and maturation to obtain confluent cells occurs in seven days of culture. Moreover, a low cell density (5.10⁴ cells/cm²) was sufficient to obtain a monolayer of confluent cells.

In contrast, the deposition of a single layer of PAH polyelectrolyte on ePTFE is not sufficient for observing similar cell viability after an identical culture time.

### 3.2.2. Cell Morphology by Scanning Electron Microscopy (SEM)

For observation with the electron microscope (STEREOSCAN S 240, CAMBRIDGE (UK)), the cells must be fixed. After washing twice with PBS buffer heated to 37° C., the cells are fixed with 2.5% glutaraldehyde, and stored at 4° C. before observation with the SEM. The samples are then prepared to permit observation in electron microscopy (dehydration, fixation and covering with a layer of gold-palladium). This investigation was carried out in the Electron Microscopy Laboratory (Pr Folliguet, Medical Faculty, Nancy).

FIGS. 6A, 6B, 6C, 6D and 6E show that, after 7 days of culture:
the endothelial cells sown on the ePTFE substrate (control) are not very numerous and have a round appearance, representative of poor spread and of poor adhesion of the cells on this substrate (FIGS. 6A and 6B),
the endothelial cells sown on a PAH polyelectrolyte deposited on the ePTFE substrate only rarely display good spread (FIGS. 6C and 6D) and the number of adherent cells is small,
the endothelial cells sown on the PEI-(PSS-PAH)₃ polyelectrolyte multilayer film deposited on the ePTFE substrate are numerous and spread out, indicating that they are adherent to the multilayer film; a confluent monolayer of endothelial cells was obtained in less than seven days of culture; cell density is high and it is difficult to distinguish the cells from one another; uniform distribution is observed.

### 3.2.3. Characterization of the Endothelial Cells

The phenotype of the endothelial cells is evaluated by expression of the von Willebrand factor (vWf) in confocal microscopy. For visualization of each cell, the nuclei are labelled with propidium iodide. After 7 days of culture, the endothelial cells are washed with DMEM (Dulbecco's Modified Eagle Medium) without phenol red (Gibco BRL, France) at 37°. They are then fixed immediately with PAF (paraformaldehyde) 1% v/v in PBS. After 10 minutes at room temperature, the PAF is removed and the cells are permeabilized using Triton-X100 (Sigma, France) at 0.5% in PBS. The cells are then incubated for 45 minutes with a mouse vWf anti-human monoclonal antibody (clone F8/86, Dako, Trappes, France) diluted 1/50 in Triton 0.1%. The cells are then washed with DMEM to remove the excess antibodies and are incubated for 30 minutes with a IgG anti-mouse polyclonal antibody conjugated with Alexa Fluor 488 (Molecular Probes, Oregon, USA) diluted 1/100 in DMEM. The isotypic control is prepared in the same conditions. The cells are incubated for 30 minutes with propidium iodide (PI) (λexcitation=535 nm, λemission=617 nm, Molecular Probes, 1 mg/mL in distilled water) diluted 1/1000 in DMEM. The labelled cells are then visualized in the confocal laser scanning microscope using an objective 40 and an He—Ne laser for the 543 nm excitation (PI) and an Ar laser for the 488 nm excitation (vWf).

FIG. 7 shows that all the cells that adhere to the PEI-(PSS-PAH)₃ polyelectrolyte multilayer film deposited on the ePTFE substrate express the vWF factor, characteristic of endothelial cells, after 7 days of culture. These observations show that there is no de-differentiation: the cells have conserved their endothelial functionality.

### Example 4

### Proliferation of Endothelial Cells on Arteries on Which a (PAH-PSS)₃-PAH Polyelectrolyte Multilayer Film was Deposited

### 4.1. Materials Used

### Composition of the Culture Medium for the Endothelial Cells

The medium used is that described in paragraph 1.1.3. “Composition of the complete medium” above.

### Cells Used

The cells used are those described in paragraph 3.1.2. “cells used”

### 4.2. Culture of Endothelial Cells Obtained From the Umbilical Vein

An umbilical cord is put in a sterile Petri dish. The exterior of the cord is cleaned with 70° ethanol. The orifice of the umbilical vein is located with forceps in order to insert a sterile tap. To remove the blood from the umbilical vein, the latter is washed three times with HBSS buffer (Hank's Balanced Salt Solution). The umbilical vein is then filled with 15 to 20 mL of a digesting solution preheated to 37° C. (trypsin/EDTA 0.25%). The cord, immersed in HBSS buffer, is put on a water bath. After incubation for 12 min, the digesting solution is collected in a 50 mL bottle containing 5 mL of complete medium. The vein is washed with HBSS buffer. The cellular suspension is centrifuged at 300 g for 10 min at room temperature. The cellular pellet is resuspended in 10 mL of HBSS buffer. After the second washing, the cells are resuspended in 5 mL of complete medium. The endothelial cells (HUVEC) are sown in a 25 cm² culture bottle, and then are put in an incubator at 37° C. (5% CO₂ and 95% air).

### 4.3. Endothelialization of the Arteries

When the endothelial cells reach confluence, the culture medium is removed and the cells are washed twice with 5 mL of HBSS buffer without Ca²⁺ or Mg²⁺. This washing makes it possible on the one hand to remove the serum, which inhibits the enzymatic activity of the trypsin, and on the other hand to release Ca²⁺ ions, which in their turn facilitate detachment of the cells. The cells are then detached using 5 mL of solution of Trypsin-EDTA 0.125%. The action of the trypsin is stopped after 2 min at 37° C. by adding 10 mL of complete medium. The cellular suspension is collected in a sterile 50-mL Falcon tube, then centrifuged at 300 g. The cellular pellet is resuspended in complete medium.

At the second passage (P1), the cells are then sown at a cell density of 10⁵ cells/cm² in the various matrices (arteries on which a (PAH-PSS)₃-PAH polyelectrolyte multilayer film was deposited and control arteries). For better distribution of the cells, the endothelialized substitutes are put in sterile Falcon tubes, stirring gently for 4 hours. They are cultured in an incubator at 37° C., 5% CO₂ for 7 days.

### 4.4. Results

### 4.4.1. Monitoring of the Phenotype by Histology

FIGS. 8A to 8D show that the endothelial cells cover the entire surface of the lumen of the re-endothelialized artery on which a (PAH-PSS)₃-PAH polyelectrolyte multilayer film was deposited or not. FIGS. 8C and 8D show maintenance of the endothelial phenotype after endothelialization.

### 4.4.2. Evaluation of the Spread by Electron Microscopy

FIGS. 9A to 9C show that the spread of the endothelial cells sown on the artery on which the (PAH-PSS)₃-PAH (9B) polyelectrolyte multilayer film was deposited is similar to the control (fresh artery 9C) and is better than that on the artery on which no multilayer film was deposited (9A).

### Example 5

### Cell Retention After Flow

### 5.1. Description of the Experiment

The retention of the HUVECs sown in the lumen of the arteries is evaluated in a flow chamber developed in the laboratory. The endothelial cells are exposed to laminar flows of 1 Pa (10 dynes/cm²), for one hour.

A peristaltic pump (Ismatech, Switzerland) provides circulation of the culture medium. Upstream of the chamber, two syringes are added to the circuit, for creating a sinusoidal modulation in order to dampen the parasitic fluctuations of the flow. A gas mixture (5% CO₂ and 95% air) is introduced into the reservoir of the medium to control the variations in pH. The system is put in a stove set to 37° C.

The shear stress is calculated from the following equation:

τ=4Qμ/πr³
τ=shear stress (Pa)
μ=viscosity of the complete medium 0.9.10⁻³ Pa·s at 37° C.
Q=flow rate (m³/s).
r=radius (m).

Consequently, knowing the value of the flow rate of the peristaltic pump, it is possible to find the shear stress exerted in the lumen of the artery. The peristaltic pump was calibrated by measuring the flow rate as a function of the rotary speed.

Following this calibration, the following relation was obtained by linear regression:
Flow rate (cm³/s)=0.458e⁻³ rotary speed (graduation)+7.049e⁻⁴
permitting precise selection of the shear stress applied.

### 5.2. Results

### 5.2.1. Evaluation of Cell Retention by Confocal Laser Scanning Microscopy

FIGS. 10A to 10D show that, after subjecting the arteries to a shear stress of 1 Pa for one hour, detachment of the endothelial cells is observed on the endothelialized arteries on which no polyelectrolyte multilayer film was deposited (Arrows). In contrast, for the arteries on which a (PAH-PSS)₃-PAH polyelectrolyte multilayer film was deposited, the layer of endothelial cells is still present.

### 5.2.2. Evaluation of Cell Retention by Electron Microscopy

FIGS. 11A to 11D show that, after subjecting the arteries to a shear stress of 1 Pa for one hour, detachment of the endothelial cells is observed on the endothelialized arteries on which no polyelectrolyte multilayer film was deposited (Arrows). In contrast, for the arteries on which a (PAH-PSS)₃-PAH polyelectrolyte multilayer film was deposited, the layer of endothelial cells is still present. Moreover, the junctions between the cells are no longer visible, which indicates that the spread of the endothelial cells is very good.

### 5.2.3. Conclusion

The results in FIGS. 10A to 10D and 11A to 11D show that the retention of the endothelial cells sown on the internal surface of the arteries on which a (PAH-PSS)₃-PAH polyelectrolyte multilayer film was deposited is better than that of the endothelial cells sown on the internal surface of the arteries on which no multilayer film was deposited.

### Example 6

### Evaluation In Vivo of the Arterial Substitutes

In this example, vascular substitutes (umbilical arteries) treated with a (PAH-PSS)₃ polyelectrolyte multilayer film are evaluated in an animal (the rabbit). The untreated de-endothelialized arteries are used as control.

### 6.1. Description of the Experiment

### 6.1.1. Animals

All the experiments carried out on the rabbit were conducted observing the current European rules on bioethics (Decree No. 2001-131 of 6 February 2001, linked to European Directive 86-609-EEC of 1986). Thus, the animals (male New Zealand white rabbits, weighing 3±0.25 kg) are of controlled origin (CEGAV, St Mars d'Egrenne, France), and were kept in an approved animal house and all necessary precautions were taken to avoid any suffering of the animal during the experiments.

### 6.1.2. Anaesthesia

Induction of anaesthesia is performed via the external marginal vein of the ear, by means of an intravenous catheter (Salva epicranial set, COOPER, Rhône-Poulenc Rorer, Melun, France), by slow injection of a dose of 40 mg/kg of pentobarbital sodium (Ceva Santé Animale, France), diluted to a quarter in physiological serum (NaCl 0.9% Cooper, Rhône-Poulenc, France). In contrast to the volatile anaesthetics, pentobarbital sodium does not seem to alter the behaviour of the polynuclear neutrophils nor of the platelets. The efficacy of anaesthesia is verified before commencement of any surgery by interdigital pinching of the rabbit's hindpaw. Anaesthesia is maintained by intravenous injection (marginal vein of the ear) of pentobarbital diluted to ¼ in physiological serum repeatedly.

### 6.1.3. Surgery

The anaesthetized animal is placed in dorsal recumbency on the heated table and its body temperature is maintained at a constant 37° C. The areas for surgical intervention are shaved and then disinfected with iodinated polyvidone (Bétadine dermique 10% ™ Laboratoire Sarget, Mérignac, France).

### 6.1.4. Catheterization of the Femoral Artery for Taking Blood Samples

After local anaesthesia with Xylocaine, an incision about 3 cm long is made in the region of the fold of the right groin. The femoral artery is isolated from the nerve and the vein, then cleared and incised to introduce a polyethylene catheter, with inside diameter of 0.58 mm and outside diameter of 0.96 mm, filled with heparinized physiological serum. This catheter is advanced about 1 cm and makes it possible to take 50 mL of blood, collected in previously heparinized 20 mL syringes.

The wound is cleaned with iodinated polyvidone and the skin is sutured with polyglactine 2-0 thread (Vicryl, Ethicon). The animal is then returned to the animal house in the conditions described previously.

### 6.1.5. Implantation of the Arterial Substitutes

On an animal previously anaesthetized, supplementary local anaesthesia with Xylocaine (AstraZeneca, Monts, France) is applied, then an incision of about 4 cm is made in the neck, along the trachea. The right carotid artery is exposed. 300 U/mL of heparin sodium (Sanofi synthelabo, France) is administered intravenously just before fitting the vascular clamps (proximal and distal level).

After clamping the carotid, an arteriotomy (0.5 cm) is made proximally, at a distance of about 1 cm from the clamp, then distally, for inserting the vascular graft there by termino-lateral bypass. Anastomosis is performed by means of vascular threads 8-0. Once the graft is in place, the carotid artery is ligatured and blood circulation in the graft is verified.

### 6.1.6. Monitoring of the Arterial Substitutes Over Time

The arterial substitutes are monitored for up to 3 months. The permeability of the substitutes is verified by Echo-Doppler. This apparatus measured the blood flow as well as the variation in diameter of the substitutes.

### 6.1.7. Euthanasia and Removal of the Arterial Substitutes

After the substitute has been in place for 1 and 12 weeks, the grafts and the control carotids (left) are removed, rinsed carefully with heparinized physiological saline solution, and then submitted to macroscopic and microscopic examination.

The animals are euthanized by injection of a lethal dose of pentobarbital sodium, according to the recommendations published by the European Commission (decree No. 2001-131 of 6 February 2001, linked to European Directive 86-609-EEC of 1986). The death of the animal is confirmed after respiratory and cardiac arrest.

### 6.2. Results

### 6.2.1. Histology

Histological examination of the substitutes in FIGS. 12A to 12F shows:
obstruction of the lumen of the control arteries after 1 week of implantation,
that the arteries on which a polyelectrolyte multilayer film was deposited remain permeable after 1 week and for up to 3 months from implantation, with weekly monitoring of the passage of blood in the arteries.

### 6.2.2. Scanning Electron Microscopy

The observations with the scanning electron microscope (FIGS. 13A to 13F) show
obstruction of the lumen of the control arteries after 1 week of implantation,
that the arteries treated do not show the presence of clots after 1 week and 3 months of implantation.

### 6.2.3. Echo-Doppler

The functionality of the arterial substitutes is monitored on a conscious animal by a non-invasive technique: “echo-Doppler”. This apparatus measured the velocity of the blood as well as the variation in diameter of the arterial substitutes.

The recordings obtained (FIGS. 14A to 14C) show that the artery on which a (PAH-PSS)₃ polyelectrolyte multilayer film was deposited has good permeability. Calculation of the area-under-curve of the recordings shows that the velocity of the blood in the arterial substitutes is equal to that measured in the control carotid. Measurement of the diameter of each arterial substitute shows neither dilatation nor aneurism.

### Example 7

### EPC Stem Cells Differentiating to Endothelial Cells on a Synthetic Substrate

### 7.1. Materials

### Composition of the Culture Medium of Endothelial Progenitors:

Commercial medium: EBM-2 supplemented with a cocktail of growth factors (VEGF, hydrocortisone, hFGF, IGF, ascorbic acid, hEGF, heparin) (Single Quot®) (Clonetics, Belgium).

### 7.2. Culturing of the Endothelial Progenitors

A leukocyte fraction from the peripheral circulation was obtained after density gradient separation. A mixture of heparinized blood and PBS (phosphate-buffered saline) (10 mL of blood in 16 mL of PBS) is added gradually to 10 mL of Histopaque® 1077 (Sigma, France), then centrifuged at 400 g for 30 min. The ring of leukocytes is aspirated with a sterile Pasteur pipette and transferred to a 50-mL tube containing 10 mL of MCDB 131 (Gibco, France) supplemented with 5 U/mL of heparin sodium (Sigma, France). The tube is then centrifuged at 250 g for 10 min, the supernatant is removed and the pellet is resuspended in 10 mL of heparinized MCDB 131. This last operation is repeated three times. The pellet is then resuspended in EBM-2 culture medium supplemented with a cocktail of growth factors (Single Quot®) (Clonetics, Belgium). About 1×10⁶ EPC (endothelial progenitor cells) per cm² are cultured in a 25 cm² culture bottle treated with fibronectin (20 μg/mL) or a (PAH-PSS)₃-PAH polyelectrolyte multilayer film. The culture medium is changed after four days, which makes it possible to remove the non-adherent cells, then every other day. These cells are cultivated for 2 weeks at 37° C. and 5% CO₂.

### 7.3. Results

### 7.3.1. Phase-Contrast Microscopy

FIGS. 15A to 15F show images obtained after observation in phase-contrast microscopy and illustrate the differentiation of the endothelial progenitors into mature endothelial cells. On the glass slide on which a polyelectrolyte multilayer film was deposited, a monolayer of cells is obtained after 14 days of culture (addition of growth factors (VEGF, hydrocortisone, hFGF, IGF, ascorbic acid, hEGF, heparin) in the medium). The morphological appearance of the monolayer obtained is similar to that of the mature endothelial cells obtained from the rabbit jugular vein (JVEC). In comparison, it takes 60 days to obtain a monolayer of cells on a glass slide covered with fibronectin.

### 7.3.2. Confocal Laser Scanning Microscopy

Phenotypic characterization of the cells after culture for 14 days is carried out by observation in confocal laser scanning microscopy (FIGS. 16A to 16L). The monolayer of cells obtained on the polyelectrolyte multilayer film corresponds well to a monolayer of endothelial cells (PECAM-1, vWF both positive). The cells are functional as they have acquired the ability to incorporate LDLs. These cells also express actin fibres, a sign of good adhesion and good spread.

### 7.3.3. Semiquantitative Investigation of Fluorescence on Images Obtained in Confocal Microscopy

Semiquantitative investigation of fluorescence on images obtained in confocal microscopy after 14 days of culture (FIGS. 17A to 17C) confirms that:
the monolayer of cells obtained on the polyelectrolyte multilayer film corresponds well to a monolayer of endothelial cells (PECAM-1, vWF both positive);
these cells are functional as they have acquired the ability to incorporate LDLs;
the endothelial cells derived from the EPCs sown on a layer of fibronectin after 14 days of culture have not proliferated so well as the endothelial cells derived from the EPCs sown on a polyelectrolyte multilayer film after 14 days of culture.

### 7.3.4. Test of Viability

FIG. 18 shows the result of a test of cell viability with Alamar Blue®. The principle and the procedure of this test were explained in example 3. FIG. 14 shows that the polyelectrolyte multilayer film has no effect on the viability of the progenitors. A significant difference is observed between the endothelial cells derived from the seeding of EPC on a polyelectrolyte multilayer film, and those derived from the seeding of EPC on fibronectin. Good metabolic activity of the cells, a sign of good cellular proliferation, is observed for the endothelial cells derived from the seeding of EPC on a polyelectrolyte multilayer film.

### Example 8

### EPC Stem Cells Differentiating to Endothelial Cells on a Natural Substrate

### 8.1. Seeding of Progenitor Endothelial Cells in the Lumen of the Arterial Matrices Reagent:

Trypan Blue (Sigma, France).
Swinging agitator generating slow rocking movements (APELEX, France).
EBM-2 culture medium supplemented with growth factors (Clonetics, Belgium).

EPCs derived from rabbit peripheral blood are recovered and the cells are counted on a Thoma cell. The viability is estimated according to the Trypan Blue exclusion test (Sigma, France). One volume of the final solution of Trypan blue is added to an equal volume of cellular suspension. The cells not allowing entry of the dye are considered to be alive.

The cellular suspension is adjusted and injected in the various matrices (arteries on which a monolayer of (PAH-PSS)₃-PAH polyelectrolytes was deposited, and the control arteries) (with a length of 4 cm); the cell density is 1×10⁷ cells/cm². To allow better distribution of the cells, the endothelialized substitutes are placed in sterile Falcons, with gentle agitation for 4 hours.

The arteries are then put in culture bottles (one artery per bottle) and are put in an incubator at 37° C. (5% CO₂ and 95% air). The culture time is one week.

### 8.2. Mechanical Stimulation: Differentiation Under Shear Stresses

To evaluate the retention of the EPCs sown in the lumen of the arteries (on which a polyelectrolyte multilayer film was deposited, and controls (without multilayer film)) and to improve their differentiation, the latter were exposed to laminar flow. The latter makes it possible to generate shear stresses of 0.1 to 0.25 Pa.

This study is carried out in a flow chamber developed in the laboratory (FIG. 19). The exposure time is 48 hours.

### Description of the Circuit:

A reservoir of complete culture medium.
A peristaltic pump (Ismatech, Switzerland).
An air inlet.
Tubes made of Pharmed® as well as autoclaveable connectors (Bioblock, France).
Flow chamber.

A peristaltic pump permits circulation of the culture medium without growth factors. Upstream of the chamber, two syringes are added to the circuit, to create sinusoidal modulation in order to dampen the parasitic fluctuations of the flow. A gas mixture (5% CO₂ and 95% air) is introduced in the reservoir of the medium to control the variations in pH. The system is put in a stove set to 37° C.

The shear stress is calculated from the following equation:

τ=4Qμ/πr³

τ: shear stress (Pa), μ: viscosity of the complete medium 0.9.10⁻³ Pa·s at 37° C.,
Q: flow rate (m³/s), r: radius (m).

Consequently, knowing the value of the flow rate of the peristaltic pump, it is possible to find the shear stress exerted in the lumen of the artery. The peristaltic pump was calibrated by measuring the flow rate as a function of the rotary speed.

The flow rate of the peristaltic pump is calibrated by the graduation of the rotary speed (FIG. 20). The relation:

y=0.0018 x
was obtained by linear regression. It allows precise selection of the shear stress applied.

### 8.3. Evaluation of the Retention of the Mature Endothelial Cells on the Substrate

The retention of the mature endothelial cells on the substrate is evaluated by:
counting the cells in the medium,
evaluating their presence on the matrix by scanning electron microscopy,
verification of the phenotype by confocal microscopy (labelling of PECAM 1, vWF, VEGFR2 and incorporation of the LDLs, as in paragraph 7.3.2.)

### Example 9

### Human Mesenchymal Stem Cells Differentiating to Endothelial Cells on a Synthetic Substrate

### 9.1. Materials

The culture substrates are glass slides:
covered with fibronectin (density: 5 μg/well),
on which a PEI-(PSS-PAH)₃ or (PSS-PAH)₂-PAH multilayer film was deposited,
covered with gelatin (at 1%, 500 μL/well)

The culture medium is as follows: alpha MEM+0.5% or 2% of SVF

### 9.2. Culturing of Human Mesenchymal Stem Cells and Differentiation

Human mesenchymal stem cells are cultured at a seeding density of 5.10³ cells per cm².

The methods of differentiation into endothelial cells are:
with or without VEGF growth factor (50 ng/mL),
in static conditions or with shearing.

The incubator is at 37° C., under 5% CO₂.

The shear stresses in the flow chamber are 0.5 Pa, 1 Pa, 1.5 Pa, 2 Pa for 24 h, 48 h, 72 h or 96 h beginning at 7 days of culture (culture time after which the cells are confluent).

### 9.3. Results

The quality of the differentiated endothelial cells can be verified:
by counting the possible presence of cells in the medium, which represent the cells that were detached or are poorly adherent), or,
by viability testing (as in paragraph 7.3.4.).

The angiogenic potential can be evaluated by
release of NO and PGI2 (prostaglandin),
incorporation of DiI-acLDL (function associated with endothelial cells as only they absorb it)
binding to lectin UEA-1 (Ulex europaeus 1): specific lectin of the endothelial cells
labelling of the actin filaments (confocal)
studying the morphology in fluorescence microscopy and confocal microscopy,
determination of the expression of VE-cadherin (CD144), von Willebrand factor (vWF) and PECAM-1 (CD31) by flow cytometry and confocal microscopy,
RT-PCR: genetic expression of the endothelial markers: VE-cadherin, VEGFR2/R1, CD31, vWF.

1-20. (canceled)

21. A method for covering
of polyelectrolyte multilayer films
or of collection of biological or biologically active molecules,
with adherent, viable and confluent cells resulting from the proliferation of initial cells,
* the aforesaid covering being obtained after a period not exceeding one month, notably 14 days after contacting the initial cells,
with the polyelectrolyte multilayer film
or with the collection of biological or biologically active molecules,
* the aforesaid covering comprising a method for proliferation of initial, stem or differentiated cells, brought in contact
with the polyelectrolyte multilayer film
or with the collection of biological or biologically active molecules coating the aforesaid polyelectrolyte multilayer film,
provided that said stem cells are not human embryonic stem cells, wherein the polyelectrolyte multilayer films is deposited on a substrate and said multilayer films:
is optionally coated, partially or completely, with a collection of biological or biologically active molecules,
and/or optionally comprise biological or biologically active molecules, incorporated between at least two adjacent layers of the aforesaid polyelectrolyte multilayer films, the incorporation being such that neither the properties of the polyelectrolyte multilayer film, nor the possible biological properties of said molecules are altered,

22. Method according to claim 21, characterized in that the initial cells are differentiated cells or stem cells,
said differentiated cells notably being selected from keratinocytes, chondrocytes, nerve cells, dendritic cells, endothelial cells, fibroblasts, epiblasts, myoblasts, cardiomyoblasts, myocytes, epithelial cells, osteocytes, osteoblasts, hepatocytes and cells of the islets of Langerhans, and,
said stem cells notably being selected from totipotent, pluripotent and multipotent cells.

23. Method according to claim 21, characterized in that the polyelectrolyte multilayer films:
* comprise or are constituted of layers, preferably alternating, of polycations and of polyanions,
said polycations notably being selected from polyallylamine (PAH), polyethyleneimine (PEI), polyvinylamine, polyaminoamide (PAMAM), polyacrylamide (PAAm), polydiallyldimethylammonium chloride (PDAC), positively charged polypeptides such as polylysine and positively charged polysaccharides such as chitosan,
and said polyanions notably being selected from polyacrylic acid (PAA), polymethacrylic acid (PMA), polystyrene sulphonic acid (PSS or SPS), negatively charged polypeptides such as polyglutamic acid and polyaspartic acid and negatively charged polysaccharides such as hyaluronan and alginate,
* and are in particular selected from (PAH-PSS)₃, (PAH-PSS)₃-PAH and PEI-(PSS-PAH)₃.

24. Method according to claim 21, characterized in that the number of layers of polyelectrolyte multilayer films is from 3 to 100, in particular 3 to 50, notably 5 to 10 and in particular 7.

25. Method according to claim 21, characterized in that the substrate is a synthetic substrate or a natural substrate,
said synthetic substrate notably being selected from glass, TCPS (“treated cell culture” polystyrene), polysiloxane, perfluoroalkyl polyethers, biocompatible polymers especially Dacron®, polyurethane, polydimethylsiloxane, polyvinyl chloride, Silastic®, polytetrafluoroethylene (ePTFE), and any material used for prostheses and/or implanted systems,
said natural substrate notably being selected from blood vessels, veins, arteries, notably decellularized umbilical arteries, said vessels, veins and arteries being obtained from organs of donors or of animals, the placental dermis, the bladder and any other substrate (organ) of human or animal origin.

26. Method according to claim 21, for the preparation of vascular endoprostheses, balloons for angioplasty, arteries or artificial vessels for grafts, vascular shunts, heart valves, artificial components for the heart, pacemakers, ventricular assist devices, catheters, contact lenses, intraocular lenses, matrices for tissue engineering, biomedical membranes, dialysis membranes, membranes for cell encapsulation, prostheses for cosmetic surgery, orthopaedic prostheses, dental prostheses, dressings, sutures, diagnostic biosensors.

27. Method of covering initial cells, stem cells or differentiated cells in vitro, comprising:
bringing initial cells in contact with polyelectrolyte multilayer films deposited on a substrate, said multilayer films being optionally coated with a collection of biological or biologically active molecules and/or optionally containing biological or biologically active molecules, incorporated between at least two adjacent layers of the aforesaid polyelectrolyte multilayer films, the incorporation being such that neither the properties of the polyelectrolyte multilayer film, nor the possible biological properties of said molecules are altered, in media permitting the proliferation of said initial cells,
proliferation of the aforesaid initial cells,
obtaining, after a period not exceeding one month, notably 14 days after the aforesaid contacting, covering of the aforesaid multilayer films or of the aforesaid collection of molecules coating the multilayer films, with adherent, viable and confluent cells resulting from the proliferation of the aforesaid initial cells,
optional recovery of said adherent, viable and confluent cells
provided that said stem cells are not human embryonic stem cells.

28. Method according to claim 27, characterized in that the method is a method of covering initial stem cells comprising:
bringing initial stem cells in contact with polyelectrolyte multilayer films deposited on a substrate in media permitting the proliferation of said initial cells,
proliferation of the aforesaid stem cells,
maturation and differentiation of the aforesaid stem cells into differentiated cells,
proliferation of the aforesaid differentiated cells derived from the aforesaid initial cells,
obtaining, after a period not exceeding one month, notably fourteen days after the aforesaid contacting, covering of the aforesaid multilayer films with adherent, viable and confluent cells resulting from the proliferation of the aforesaid initial cells,
optional recovery of said adherent, viable and confluent cells.

29. Method according to claim 27, characterized in that the method is a method of covering differentiated initial cells comprising:
bringing differentiated initial cells in contact with polyelectrolyte multilayer films deposited on a substrate in media permitting the proliferation of said initial cells,
proliferation of the aforesaid differentiated cells,
obtaining, after a period not exceeding one month, notably fourteen days and in particular seven days after the aforesaid contacting, covering of the aforesaid multilayer films with adherent, viable and confluent cells resulting from the proliferation of the aforesaid initial cells,
optional recovery of said adherent, viable and confluent cells.

30. Method according to claim 27, characterized in that the method comprises:
bringing initial cells in contact with polyelectrolyte multilayer films deposited on a substrate, said multilayer films being optionally coated with a collection of biological or biologically active molecules, and/or optionally containing biological or biologically active molecules, incorporated between at least two adjacent layers of the aforesaid polyelectrolyte multilayer films, the incorporation being such that neither the properties of the polyelectrolyte multilayer film, nor the possible biological properties of said molecules are altered, in media permitting the proliferation of said initial cells,
proliferation of the aforesaid initial cells,
obtaining, after a period not exceeding one month, notably 14 days after the aforesaid contacting, covering of the aforesaid multilayer films or of the aforesaid collection of biological or biologically active molecules coating the multilayer film, with adherent, viable and confluent cells resulting from the proliferation of the aforesaid initial cells,
recovery of said adherent, viable and confluent cells.

31. Method of covering of endothelial initial cells according to claim 27 comprising:
bringing endothelial initial cells in contact with polyelectrolyte multilayer films selected from (PAH-PSS)₃(PAH-PSS)₃-PAH and PEI-(PSS-PAH)₃, deposited on a substrate, notably a natural substrate such as a blood vessel or a decellularized artery, or a biocompatible synthetic substrate having the shape of a vessel or artery, said multilayer films being optionally coated with a collection of biological or biologically active molecules, and/or optionally containing biological or biologically active molecules, incorporated between at least two adjacent layers of the aforesaid polyelectrolyte multilayer films, the incorporation being such that neither the properties of the polyelectrolyte multilayer film, nor the possible biological properties of said molecules are altered, in media permitting the proliferation of the aforesaid endothelial initial cells,
proliferation of the aforesaid endothelial initial cells,
obtaining, after a period not exceeding one month, notably 14 days and in particular 7 days after the aforesaid contacting, covering of the aforesaid multilayer films or of the aforesaid collection of biological or biologically active molecules coating the multilayer films, with adherent, viable and confluent endothelial cells resulting from the proliferation of the aforesaid endothelial initial cells.

32. Method of covering of initial stem cells according to claim 27, comprising:
bringing initial stem cells in contact with polyelectrolyte multilayer films selected from (PAH-PSS)₃, (PAH-PSS)₃-PAH and PEI-(PSS-PAH)₃, deposited on a substrate, said multilayer films being optionally coated with a collection of biological or biologically active molecules, and/or optionally containing biological or biologically active molecules, incorporated between at least two adjacent layers of the aforesaid polyelectrolyte multilayer films, the incorporation being such that neither the properties of the polyelectrolyte multilayer film, nor the possible biological properties of said molecules are altered, in media permitting the proliferation of the aforesaid initial stem cells,
proliferation of the aforesaid initial stem cells,
maturation and differentiation of the aforesaid stem cells into endothelial cells,
proliferation of the aforesaid endothelial cells derived from the aforesaid initial stem cells,
obtaining, after a period not exceeding 14 days after the aforesaid contacting, covering of the aforesaid multilayer films or of the aforesaid collection of biological or biologically active molecules coating the multilayer films, with adherent, viable and confluent endothelial cells resulting from the proliferation of the aforesaid initial stem cells.

33. Method according to claim 27, characterized in that said initial stem cells are notably selected from totipotent, pluripotent and multipotent cells.

34. Method according to claim 27, characterized in that said differentiated initial cells are notably selected from keratinocytes, chondrocytes, nerve cells, dendritic cells, endothelial cells, fibroblasts, epiblasts, myoblasts, cardiomyoblasts, myocytes, epithelial cells, osteocytes, osteoblasts, hepatocytes and cells of the islets of Langerhans.

35. Method according to claim 27,
characterized in that said polyelectrolyte multilayer films
* are constituted of layers, preferably alternating, of polycations and of polyanions,
the polycations notably being selected from polyallylamine (PAH), polyethyleneimine (PEI), polyvinylamine, polyaminoamide (PAMAM), polyacrylamide (PAAm), polydiallyldimethylammonium chloride (PDAC), positively charged polypeptides such as polylysine and positively charged polysaccharides such as chitosan,
and the polyanions notably being selected from polyacrylic acid (PAA), polymethacrylic acid (PMA), polystyrene sulphonic acid (PSS or SPS), negatively charged polypeptides such as polyglutamic acid and polyaspartic acid and negatively charged polysaccharides such as hyaluronan and alginate, and,
* are in particular selected from (PAH-PSS)₃, (PAH-PSS)₃-PAH and PEI-(PSS-PAH)₃.

36. Composition comprising:
a substrate,
polyelectrolyte multilayer films deposited on said substrate, said multilayer films being optionally coated with a collection of biological or biologically active molecules and/or optionally containing biological or biologically active molecules, incorporated between at least two adjacent layers of the aforesaid polyelectrolyte multilayer films, the incorporation being such that neither the properties of the polyelectrolyte multilayer film, nor the possible biological properties of said molecules are altered, and
a layer of stem cells covering said polyelectrolyte multilayer films, provided that said stem cells are not human embryonic stem cells.

37. Composition comprising:
a natural substrate,
polyelectrolyte multilayer films deposited on said substrate, said multilayer films being optionally coated with a collection of biological or biologically active molecules and/or optionally containing biological or biologically active molecules, incorporated between at least two adjacent layers of the aforesaid polyelectrolyte multilayer films, the incorporation being such that neither the properties of the polyelectrolyte multilayer film, nor the possible biological properties of said molecules are altered, and
a layer of differentiated cells covering said polyelectrolyte multilayer film.

38. Composition comprising:
a substrate,
polyelectrolyte multilayer films deposited on said substrate, said multilayer films being coated with a collection of biological or biologically active molecules, and/or containing biological or biologically active molecules, incorporated between at least two adjacent layers of the aforesaid polyelectrolyte multilayer films, the incorporation being such that neither the properties of the polyelectrolyte multilayer film, nor the possible biological properties of said molecules are altered, and
a layer of differentiated cells covering said biological or biologically active molecules.

39. Composition according to claim 36, characterized in that said polyelectrolyte multilayer films
* are constituted of layers, preferably alternating, of polycations and of polyanions,
the polycations notably being selected from polyallylamine (PAH), polyethyleneimine (PEI), polyvinylamine, polyaminoamide (PAMAM), polyacrylamide (PAAm), polydiallyldimethylammonium chloride (PDAC), positively charged polypeptides such as polylysine and positively charged polysaccharides such as chitosan,
and the polyanions notably being selected from polyacrylic acid (PAA), polymethacrylic acid (PMA), polystyrene sulphonic acid (PSS or SPS), negatively charged polypeptides such as polyglutamic acid and polyaspartic acid and negatively charged polysaccharides such as hyaluronan and alginate,
* and are in particular selected from (PAH-PSS)₃, (PAH-PSS)₃-PAH and PEI-(PSS-PAH)₃.

40. Composition according to claim 36, characterized in that said substrate is a natural or synthetic substrate,
said natural substrate notably being selected from blood vessels, veins, arteries, notably decellularized umbilical arteries, said vessels, veins and arteries being obtained from organs of donors or of animals, the placental dermis, the bladder and any other substrate (organ) of human or animal origin,
said synthetic substrate notably being selected from glass, TCPS (“treated cell culture” polystyrene), polysiloxane, perfluoroalkyl polyethers, biocompatible polymers especially Dacron®, polyurethane, polydimethylsiloxane, polyvinyl chloride, Silastic®, polytetrafluoroethylene (ePTFE) and any material used for prostheses and/or implanted systems.
